(19) United States
(12) Patent Application Publication
(10) Pub. No.: US 2023/0012307 A1
(43) Pub. Date: Jan. 12, 2023

(54) PULMONARY VEIN ISOLATION CATHETERS AND ASSOCIATED DEVICES, SYSTEMS, AND METHODS

(71) Applicant: Affera, Inc., Newton, MA (US)

(72) Inventors: Doron Harlev, Brookline, MA (US); Andrew Miles Wallace, Needham, MA (US); Paul Brian Hultz, Brookline, MA (US)

(21) Appl. No.: 17/757,359

(22) PCT Filed: Dec. 16, 2020
(86) PCT No.: PCT/US20/65314

§ 371 Date: Jun. 14, 2022

Related U.S. Application Data
(60) Provisional application No. 62/948,736, filed on Dec. 16, 2019.

(51) Int. Cl. A61B 18/14

(52) U.S. Cl. CPC A61B 18/1492; A61B 2018/00577

(57) ABSTRACT

Pulmonary vein isolation catheters and associated devices, systems, and methods are disclosed herein. In some embodiments, a pulmonary vein isolation catheter includes a tip section having an expandable portion and a deployment member. The expandable portion includes a plurality of mesh electrode panels that are electrically insulated from one another. The expandable portion is mechanically coupled to (i) the deployment member at a distalmost portion of the tip section and (ii) a distal end portion of a catheter shaft. The expandable portion is expandable and compressible via proximal and distal movement, respectively, of the deployment member. In some embodiments, the expandable portion in a deployed state is pear- or onion-shaped and includes a nose portion and/or an active body portion. The nose portion can be insulated and/or configured to fit within a pulmonary vein and position the active body portion against tissue about the ostium of the pulmonary vein.

### CROSS-REFERENCE TO RELATED APPLICATIONS

This application claims the benefit of U.S. Provisional Patent Application No. 62/948,736, filed Dec. 16, 2019, which is incorporated by reference herein in its entirety.

### BACKGROUND

Atrial fibrillation is an abnormal heart rhythm characterized by rapid or irregular beating of the atrial chambers of the heart. One possible cause of atrial fibrillation is extra firings of the heart induced by pulmonary veins that carry oxygenated blood from an individual's lungs to the left atrium of the heart. Thus, a common treatment for atrial fibrillation is to electrically isolate one or more pulmonary veins from the left atrium using a catheter configured to deliver ablative energy around the ostium of the pulmonary vein(s).

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1 is a schematic representation of a system for treating a human patient and configured in accordance with various embodiments of the present technology.

FIG. 2 is a perspective view of a medical device of the system of FIG. 1 configured in accordance with various embodiments of the present technology.

FIGS. 3A and 3B are schematic representations of a tip section of the medical device of FIG. 2 configured in accordance with various embodiments of the present technology.

FIG. 4 is a schematic representation of a deployment member of the medical device of FIG. 2 configured in accordance with various embodiments of the present technology.

FIG. 5 is a top view of a distalmost portion of the tip section of the medical device of FIG. 2 configured in accordance with various embodiments of the present technology.

FIGS. 6A and 6B are schematic representations of the tip section of the medical device in a compressed state and a deployed state, respectively, in accordance with various embodiments of the present technology.

FIGS. 7A and 7B are schematic representations of a panel of a modular electrode of the tip section of the medical device of FIG. 2 and configured in accordance with various embodiments of the present technology.

FIG. 8A is an exploded view of eyelets and a corresponding rivet for connecting multiple panels of the modular electrode of the medical device of FIG. 2 and configured in accordance with embodiments of the present technology.

FIG. 8B is a schematic representation of eyelets and rivets for connecting multiple panels of the electrode of the tip section of the medical device of FIG. 2 configured in accordance with various embodiments of the present technology.

FIG. 8C is a cross-sectional view of a rivet connecting two eyelets of panels of the electrode of the tip section of the medical device of FIG. 2 configured in accordance with various embodiments of the present technology.

FIG. 8D is a schematic representation of an assembled tip section of the medical device of FIG. 2 configured in accordance with various embodiments of the present technology.

FIGS. 9A and 9B are an exploded view and a partial cross-sectional view, respectively, of a nose portion of the tip section of the medical device of FIG. 2 and configured in accordance with various embodiments of the present technology.

FIGS. 10A and 10B are an exploded view and a side view, respectively, of a proximal portion of the tip section of the medical device of FIG. 2 configured in accordance with various embodiments of the present technology.

FIGS. 11A and 11B are schematic representations of a tip section of a medical device in a deployed state and configured in accordance with various embodiments of the present technology.

FIGS. 12A and 12B are a top view and a top perspective view, respectively, of a distalmost portion of the tip section of FIGS. 11A and 11B.

FIG. 13 is a side perspective view of the tip section of FIGS. 11A-12B.

FIGS. 14A-14C are cross-sectional, side perspective, and top perspective views, respectively, of a portion of the tip section of FIGS. 11A-13 schematically illustrating how mesh electrode panels can be attached to form an expandable portion of the tip section 124 in accordance with various embodiments of the present technology.

FIG. 15 is a schematic representation of a tip section of a medical device configured in accordance with various embodiments of the present technology.

FIG. 16 is a schematic representation of a tip section of a medical device configured in accordance with various embodiments of the present technology positioned at an ostium of a pulmonary vein within an anatomical structure of a patient in accordance with various embodiments of the present technology.

FIG. 17 is a flow diagram illustrating a method for positioning a tip section of a medical device configured in accordance with various embodiments of the present technology at a treatment site within an anatomical structure of a patient in accordance with various embodiments of the present technology.

FIG. 18 is a flow diagram illustrating a method for diagnosing and/or treating tissue at a treatment site within an anatomical structure of a patient in accordance with various embodiments of the present technology.

### DETAILED DESCRIPTION

### A. Overview

As discussed above, atrial fibrillation is an abnormal heart rhythm that can be caused by extra firings of the heart induced by pulmonary veins. Thus, a common treatment for atrial fibrillation is to electrically isolate one or more pulmonary veins from the left atrium of the heart using a minimally-invasive radiofrequency, cryotherapy, or pulsed field ablation catheter. In particular, a catheter is used to deliver energy and form a lesion on the wall of the heart about the ostium of the pulmonary vein. The applied energy to cardiac tissue at the treatment site blocks the tissue's electrical activity. In turn, abnormal electrical signals in the pulmonary vein can be prevented from propagating through the blocked tissue into the heart, thereby preventing atrial fibrillation.

The inventors have realized several challenges encountered when electrically isolating a pulmonary vein from the left atrium of the heart. For example, applying energy to the wall of a pulmonary vein (as opposed to only the wall of the heart in the left atrium about the ostium of the pulmonary vein) can cause undesirable stenosis in the pulmonary vein or may fail to isolate all of the tissue responsible for atrial fibrillation. Thus, correct positioning of a catheter tip at the ostium of a pulmonary vein is important before applying energy. In addition, pulmonary veins vary in size between patients as well as across a single patient's heart. Therefore, a catheter tip should be scalable to account for the different sizes of pulmonary veins. Furthermore, as the catheter tip varies in deployment size, the effective surface area of the tip through which energy is delivered varies. As a result, current density applied via the catheter tip also varies. For this reason, granular control over the amount of energy applied through the catheter tip as well as over the portions of the tip energized to apply energy (both of which are lacking in conventional pulmonary vein isolation catheters) are required to effectively adapt energy delivery to a patient's anatomy or other treatment conditions and to avoid undesired collateral damage to the anatomy (e.g., to an individual's esophagus).

To address these challenges, the inventors have developed pulmonary vein isolation catheters having tip sections that include an expandable portion formed from several mesh electrode panels. In some embodiments, the expandable portion includes an insulated neck portion, an active modular electrode, and/or a nose portion that may or may not be insulated. The nose portion is configured to at least partially fit within a pulmonary vein and facilitate proper positioning of the modular electrode against cardiac tissue about the ostium of the pulmonary vein. Furthermore, the expandable portion can be expanded to various degrees between a fully collapsed state (e.g., to allow passage through an introducer sheath) and a fully deployed state to account for various sizes of pulmonary veins. In these and other embodiments, the mesh electrode panels that together form the expandable portion are electrically insulated from one another and are individually energizable. In this manner, pulmonary vein isolation catheters configured in accordance with the present technology are expected to provide granular control over which portion(s) of the modular electrode are used to deliver energy to tissue as well as granular control over the amount of energy delivered to regions of tissue about the modular electrode of the expandable portion.

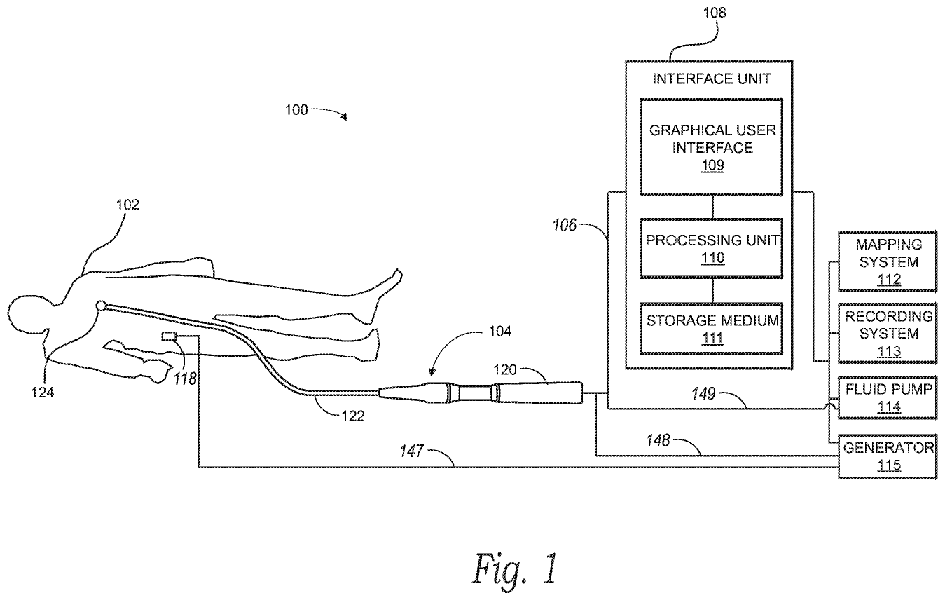

Specific details of several embodiments of the present technology are described herein with reference to FIGS. 1-18. Although many of the embodiments are described with respect to pulmonary vein isolation catheters and associated devices, systems, and methods, other applications and other embodiments in addition to those described herein are within the scope of the present technology. For example, unless otherwise specified or made clear from context, the devices, systems, and methods of the present technology can be used for any of various medical procedures, such as procedures performed on a hollow anatomical structure of a patient, and, more specifically, in procedures for stimulating, electrically isolating, or otherwise treating tissue within and/or proximal the anatomical structure. Thus, for example, the systems, devices, and methods of the present disclosure can be used as part of a medical treatment associated with diagnosis, treatment, or both of a cardiac condition (e.g., cardiac arrhythmia). Additionally, or alternatively, the devices, systems, and methods of the present disclosure can be used in one or more medical procedures associated with other interventional procedures (e.g., renal and/or carotid denervation) involving ablation of target tissue.

It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

As used herein, the term “physician” shall be understood to include any type of medical personnel who may be performing or assisting a medical procedure and, thus, is inclusive of a doctor, a nurse, a medical technician, other similar personnel, and any combination thereof. Additionally, or alternatively, as used herein, the term “medical procedure” shall be understood to include any manner and form of diagnosis, treatment, or both, inclusive of any preparation activities associated with such diagnosis, treatment, or both. Thus, for example, the term “medical procedure” shall be understood to be inclusive of any manner and form of movement or positioning of a medical device in an anatomical chamber. As used herein, the term “patient” should be considered to include human and/or non-human (e.g., animal) patients upon which a medical procedure is being performed.

### B. Selected Embodiments of Pulmonary Vein Isolation Catheters and Associated Devices, Systems, and Methods

1. Pulmonary Vein Isolation Catheter Systems

FIG. 1 is a schematic representation of a system 100 for treating a patient 102 configured in accordance with an embodiment of the present technology. In the arrangement shown in FIG. 1, the system 100 is being used to perform a medical procedure (e.g., a pulmonary vein isolation procedure) on the patient 102. The system 100 can include a medical device 104 connected via an extension cable 106 to an interface unit 108. The interface unit 108 can include a graphical user interface 109, a processing unit 110 (e.g., one or more processors), and a storage medium 111. The graphical user interface 109 and the storage medium 111 can be in electrical communication (e.g., wired communication, wireless communication, or both) with the processing unit 110. The storage medium 111 can have stored thereon computer executable instructions for causing the one or more processors of the processing unit 110 to carry out one or more portions of various methods described herein, unless otherwise indicated or made clear from context. Additionally, or alternatively, the storage medium 111 can have stored thereon computer-executable instructions for causing the processing unit 110 and/or the graphical user interface 109 to display various information collected by and/or related to the medical device 104.

A mapping system 112, a recording system 113, a fluid pump 114, and a generator 115 can be connected to the interface unit 108. The fluid pump 114 can be removably and fluidly connected to the medical device 104 via fluid line 149. The generator 115 can also, or instead, be connected to the medical device 104 via one or more wires 148 and/or be connected to one or more return electrodes 118 attached to the skin of the patient 102 via one or more wires 147. In use, electrical energy can be delivered from the generator 115 to the medical device 104 where, as described in further detail below, the electrical energy is ultimately deliverable to a tip section 124 (e.g., to a modular electrode (not shown in FIG. 1) of the tip section 124) to ablate, treat, or diagnose tissue at a treatment site. The mapping system 112 can be used prior to and/or during the medical procedure to map tissue of the patient and to determine which region or regions of tissue require treatment. The recording system 113 can be used throughout the medical procedure, as well as before or after treatment.

The medical device 104 can be any of various different medical devices known in the art (e.g., for diagnosis, treatment, or both). In the illustrated embodiments, the medical device 104 is a catheter 104 having a handle 120, a shaft 122, and a tip section 124. The tip section 124 generally includes any portion of the catheter 104 that directly or indirectly engages tissue for the purpose of treatment, diagnosis, or both and, therefore, can include all manner and type of contact and/or non-contact interaction with tissue known in the art. For example, the tip section 124 can include contact and/or non-contact interaction with tissue in the form of energy interaction (e.g., electrical energy, ultrasound energy, light energy, and any combinations thereof) and further, or instead, can include measurement of electrical signals emanating from tissue. Thus, for example, the tip section 124 can deliver energy (e.g., electrical energy) to tissue in the anatomical structure as part of any number of procedures including treatment (e.g., radiofrequency (RF) ablation, irreversible electroporation, pulsed field ablation, etc.), diagnosis (e.g., mapping), or both.

The tip section 124 and at least a portion of the shaft 122 can be inserted into an anatomical structure (e.g., a heart) of the patient 102 via a vein or artery in the patient's leg or arm. In particular, the tip section 124 can be deliverable to a treatment site (e.g., to an ostium of a pulmonary vein in the left atrium of the patient's heart) using an introducer (e.g., a steerable sheath such as an Abbott Agilis™ steerable introducer) and/or a guidewire (not shown in FIG. 1). Contrast injection and/or further advancement of the guidewire may be used in some embodiments to verify placement at the treatment site, as described in greater detail below.

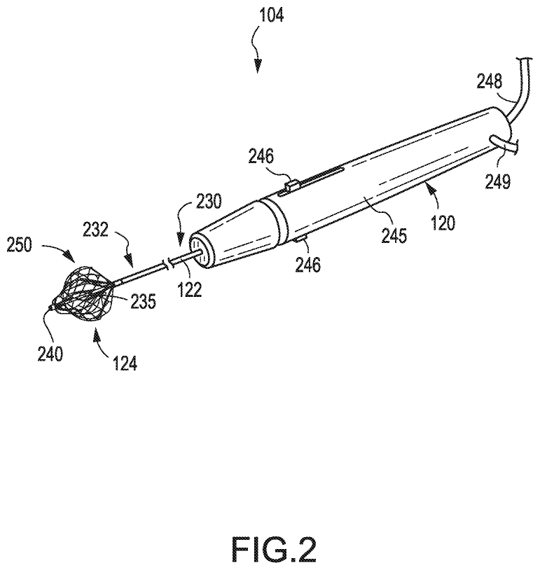

FIG. 2 is a perspective view of the catheter 104 of the system 100 of FIG. 1 configured in accordance with various embodiments of the present technology. As shown in FIG. 2, the handle 120 of the catheter 104 can be coupled to a proximal end portion 230 of the shaft 122, and the tip section 124 can be coupled to a distal end portion 232 of the shaft 122 opposite the proximal end portion 230. The tip section 124 includes an expandable portion 250 and a deployment member 235. As used herein, the terms “expandable” and “deformable” are used interchangeably, unless otherwise specified or made clear from the context. Thus, for example, it should be understood that the expandable portion 250 is deformable unless otherwise specified. The deployment member 235 extends from a distalmost portion 240 of the tip section 124 to at least the proximal end portion 230 of the shaft 122.

The shaft 122 can be formed of a number of different biocompatible materials that provide the shaft 122 with sufficient sturdiness and flexibility to allow the shaft 122 to be navigated through blood vessels of a patient. Examples of suitable materials from which the shaft 122 can be formed include polyether block amides (e.g., Pebax®, commercially available from Arkema of Colombes, France), nylon, polyurethane, Pellethane® (commercially available from The Lubrizol Corporation of Wickliffe, Ohio), and silicone. In certain implementations, the shaft 122 includes multiple different materials along its length. The materials can, for example, be selected to provide the shaft 122 with increased flexibility at the distal end, when compared to the proximal end. The shaft 122 can also, or instead, include a tubular braided element that provides torsional stiffness while maintaining bending flexibility to one or more regions of the shaft 122. Further, or in the alternative, the shaft material can include radiopaque agents such as barium sulfate or bismuth, to facilitate fluoroscopic visualization.

In these and other embodiments, the shaft 122 can define a lumen that can be in fluid communication with the fluid pump 114 (FIG. 1). For example, the shaft 122 in some embodiments defines a lumen extending from the proximal end portion 230 of the shaft 122 to the distal end portion 232 of the shaft 122. The lumen can be in fluid communication with the fluid pump 114, via the fluid line 149 (FIG. 1) and a fluid line connector 249 of the handle 120, such that fluid (e.g., saline, contrast dye, etc.) can be pumped from the fluid pump 114 to the tip section 124. Additionally, or alternatively, the shaft 122 can include electrical wires (such as any one or more of the wires 148 shown in FIG. 1) extending along the shaft 122 to carry signals between the tip section 124 and the handle 120 and/or the interface unit 108 and/or to carry electrical power (e.g., electrical energy) from the generator 115 to the tip section 124.

The handle 120 can include a housing 245 and an actuation portion 246. In use, the actuation portion 246 can be operated to extend or retract (e.g., contract) the deployment member 235 to deploy (e.g., expand, uncompress, etc.) or compress the tip section 124 of the catheter 104, as described in greater detail below. In these and other embodiments, the handle 120 can include one or more additional actuation portions (not shown), such as one or more actuation portions that can be operated to deflect the distal end portion 232 of the shaft to facilitate positioning the tip section 124 into contact with tissue at a treatment site. The handle 120 can further, or instead, be coupled to the fluid line connector 249 and/or to an electrical connector 248 for delivery of fluid and/or electrical signals (e.g., electrical energy), respectively, along the shaft 122 to/from the tip section 124.

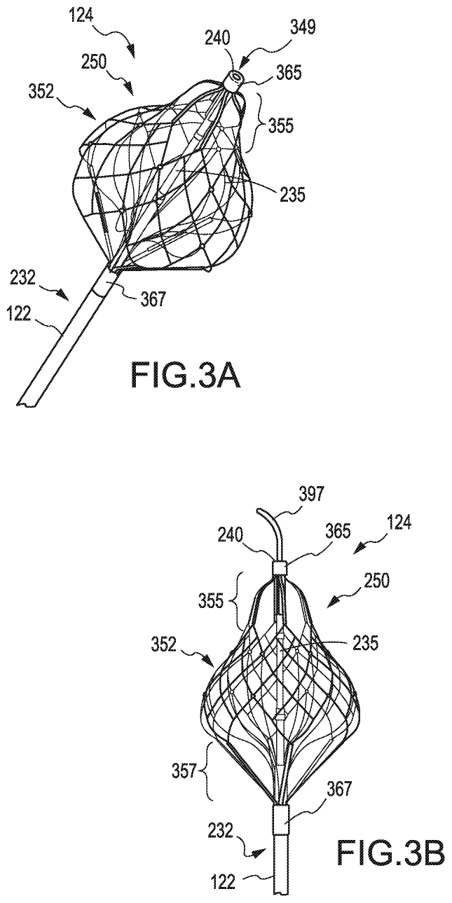

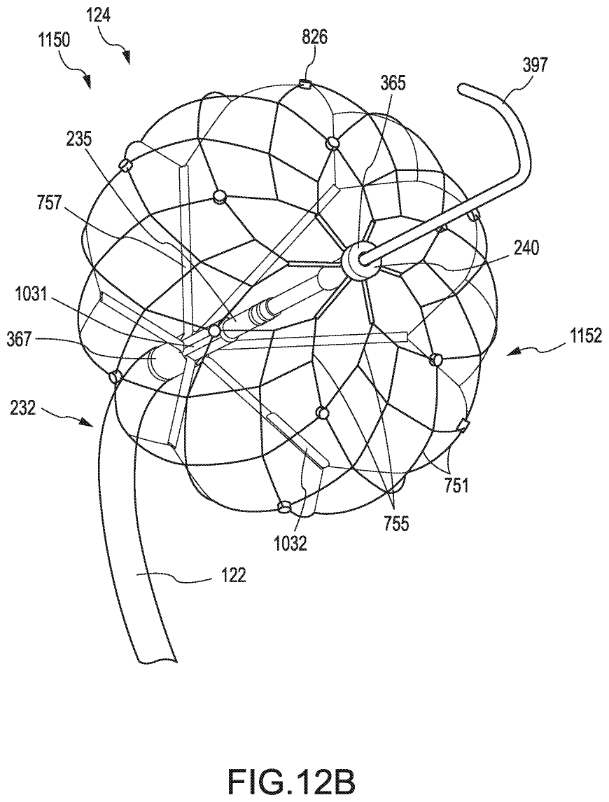

FIGS. 3A and 3B are schematic representations of the tip section 124. As shown, the expandable portion 250 of the tip section 124 can be generally “pear” shaped having a nose portion 355, a neck portion 357 (FIG. 3B), and an active body portion 352 (referred to hereinafter as a “modular electrode 352”). In other embodiments, the expandable portion 250 can have a different general shape (e.g., spherical, conical, cylindrical, hourglass, etc.). For example, as described in greater detail below with respect to FIGS. 11A-15, the expandable portion 250 of the tip section 124 can be generally “onion” shaped in some embodiments.

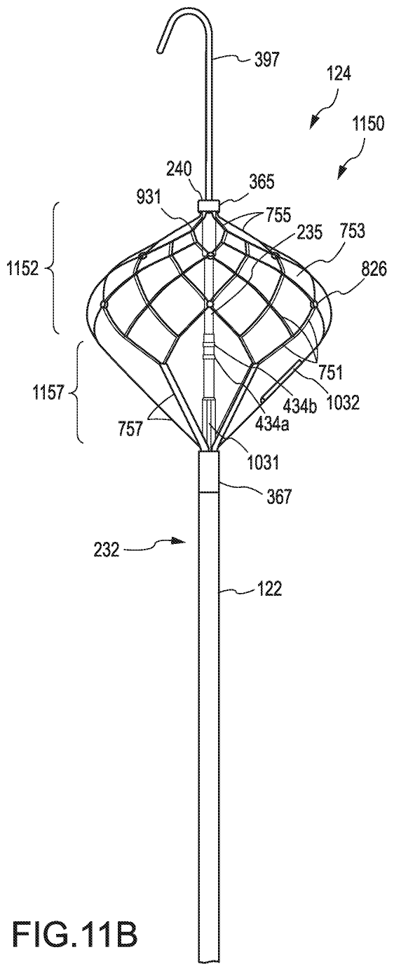

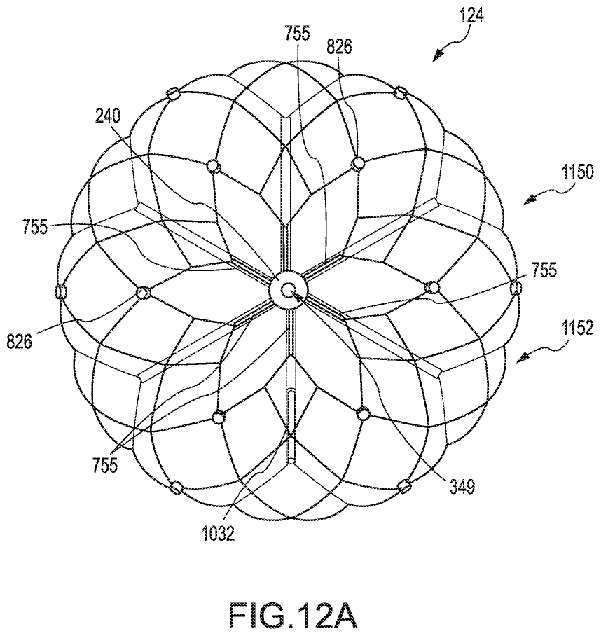

As described in greater detail below with respect to FIGS. 9A-10B, the neck portion 357 of the expandable portion 250 is coupled (e.g., mechanically coupled) to the distal end portion 232 of the shaft 122 via a coupler 367, and the nose portion 355 of the expandable portion is coupled (e.g., mechanically coupled) to the deployment member 235 via a coupler 365 at the distalmost portion 240 of the tip section 124.

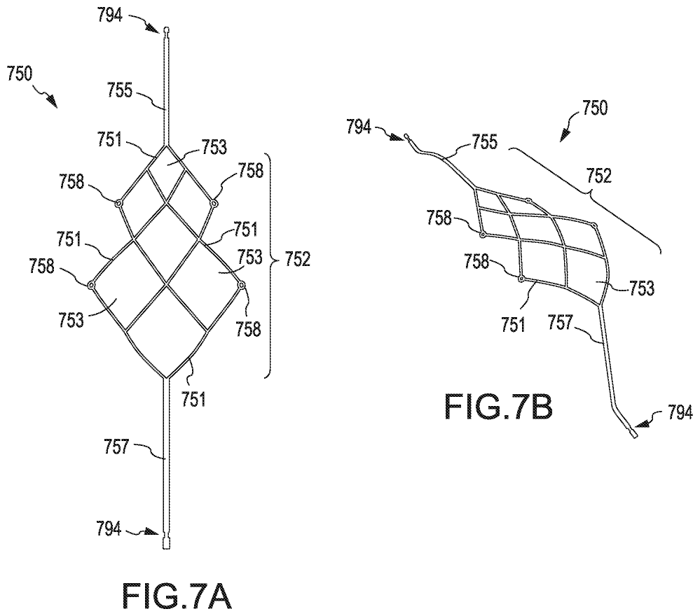

The nose portion 355 of the tip section 124 is configured to fit at least partially within a pulmonary vein of the patient 102 (FIG. 1) and facilitate proper positioning of the modular electrode 352 against cardiac tissue about the ostium of the pulmonary vein. In some embodiments, the modular electrode 352 in at least a fully deployed state (described in greater detail below with respected to FIGS. 6A and 6B) has a maximum radial dimension relative to the shaft 122 that is larger than a maximum radial dimension of the ostium of a pulmonary vein of the patient 102 (FIG. 1) to prevent all or a portion of the modular electrode 352 from being inserted within the pulmonary vein in at least the fully developed state. For example, the expandable portion 250 in the fully uncompressed state has an outer diameter of greater than about 20 mm and less than about 40 mm (e.g., a diameter between 28 mm and 30 mm, or about 29 mm).

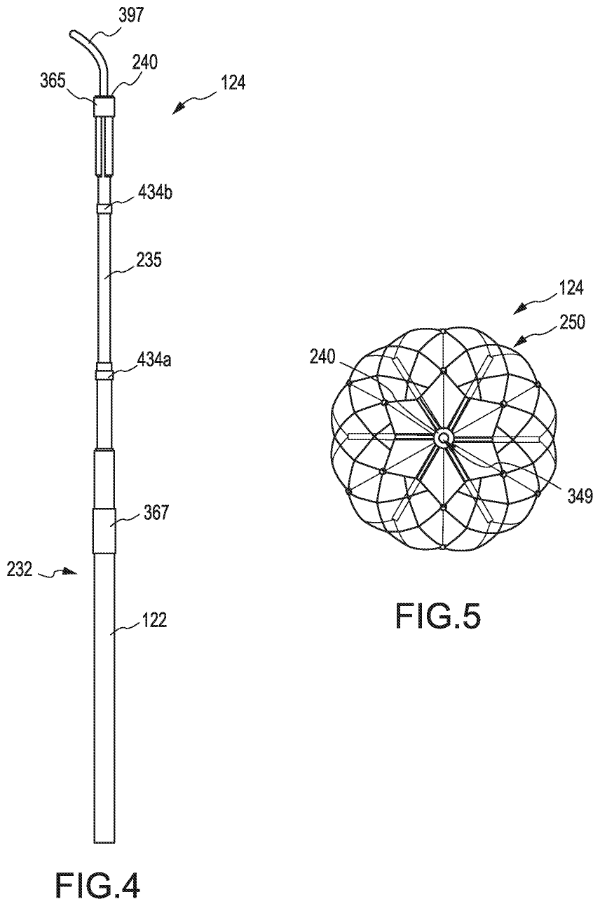

FIG. 4 is a schematic representation of the tip section 124 without the expandable portion 250 (FIGS. 3A and 3B). In particular, FIG. 4 is primarily a schematic representation of the deployment member 235. Referring to FIGS. 3A-4 together, the deployment member 235, in some embodiments, is coaxial and can be a braided polyimide tube having a polytetrafluoroethylene (PTFE) inner lining (e.g., to receive a guidewire 397). In this regard, the deployment member 235 is an elongate member that is sufficiently flexible to bend with movement of the shaft 122 while being sufficiently rigid to resist buckling, kinking, or other types of deformation in response to a force required to move, expand, or compress the tip section 124 of the catheter 104. In some embodiments, the braided polyimide tube can, additionally or alternatively, have a composite PTFE outer lining to allow smooth motion during deployment and compression of the expandable portion 250.

As best shown in FIG. 4, the deployment member 235 can include one or more ring electrodes 434. For example, the deployment member 235 can include a first ring electrode 434a positioned proximate the distal end portion 232 of the shaft 122 and/or a second ring electrode 434b positioned proximate the distalmost portion 240 of the tip section 124. In some embodiments, the ring electrodes 434a and/or 434b can be formed of platinum iridium and/or be radiopaque to facilitate fluoroscopic visualization and aid in determining a location, shape, and/or orientation of the tip section 124 of the catheter 104 while the tip section 124 is within the patient 102. Additionally, or alternatively, the ring electrodes 434a and/or 434b can be passive electrodes configured to measure electrical activity, and/or the ring electrodes 434a and/or 434b can be driven electrodes that are part of ground circuitry and/or impedance measuring circuitry, as described in greater detail below.

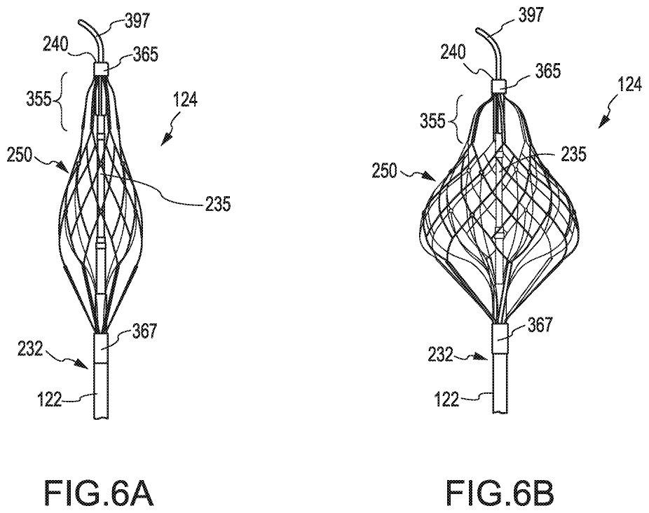

FIG. 5 is a top view of the distalmost portion 240 of the tip section 124. Referring to FIGS. 3A-5 together, the deployment member 235 can define one or more lumens. For example, the deployment member 235 can define a lumen 349 (FIGS. 3A and 5) that can receive the guidewire 397 (FIGS. 3B and 4) such that the tip section 124 of the catheter 104 may be delivered over-the-wire to a treatment site. The lumen 349 can be configured to receive various sizes of guidewires 397 (e.g., guidewires between approximately 0.030″ and approximately 0.040″, including 0.032″, 0.035″, and 0.038″). In some embodiments, the guidewire 397 can be introduced into the catheter 104 via the electrical connector 248 (FIG. 2) or the fluid connector 249 (FIG. 2) of the handle 120 (FIG. 2). In other embodiments, the catheter 104 and/or the deployment member 235 can include a separate guidewire port (not shown) through which the guidewire 397 can be introduced into the catheter 104 and/or into the deployment member 235.

In these and other embodiments, the lumen 349 and/or another lumen defined by the deployment member 235 can be in fluid communication with a fluid delivery device such as the fluid pump 114 (FIG. 1) to deliver fluid (e.g., saline, contrast dye, etc.) to at least the tip section 124 of the catheter 104. In this regard, the deployment member 235 can be configured to deliver fluid along the length of the shaft 122 to the treatment site via the tip section 124 (e.g., for irrigation (cooling) of the modular electrode 352, flushing (washing) of various components of the tip section 124, and/or positioning of the tip section 124). For example, the deployment member 235 can deliver fluid out of an opening of the lumen 349 at the distalmost portion 240 of the tip section 124. In turn, blood flowing through a pulmonary vein toward the left atrium of the patient's heart from a lung of the patient 102 (FIG. 1) can carry fluid dispersed from the opening of the lumen 349 across and/or proximate an outer portion of the modular electrode 352 in contact with tissue, thereby facilitating local heat transfer away from the outer portion of the modular electrode 352. In general, it should be appreciated that such local heat transfer can reduce the likelihood of blood clotting or charring during tissue treatment.

In these and other embodiments, the deployment member 235 can include holes (not shown) spaced axially along and/or circumferentially about the deployment member 235 at the tip section 124 to deliver fluid to the treatment site from within the expandable portion 250. In some embodiments, the holes can be cut into the deployment member 235 (e.g., using a laser) and/or formed or molded into the deployment member 235 (e.g., using a mechanical punch). The holes in the deployment member 235 can be uniformly distributed along and/or about the deployment member 235 to facilitate directing fluid toward substantially the entire inner portion of the modular electrode 352 and/or to produce a relatively uniform dispersion of fluid along the inner portion of the modular electrode 352. It should be appreciated, however, that the holes in the deployment member 235 can be distributed along and/or about the deployment member 235 in any configuration that facilitates multi-directional dispersion of fluid toward the inner portion of the modular electrode 352.

As used herein, the term “holes” should be understood to include any size and shape of discrete orifices having a maximum dimension and through which fluid can flow and, thus, should be understood to include any manner and form of substantially geometric shapes (e.g., substantially circular shapes) and, also or instead, substantially irregular shapes, unless otherwise specified or made clear from the context. The size and number of the holes defined by the deployment member 235 are selected such that the pressure of fluid in the respective lumen of the deployment member 235 is sufficient to prevent blood from entering the holes. For example, providing for some margin of variation in pressure of the fluid, the size and number of the holes defined by the deployment member 235 can be selected such that the pressure of the fluid in the deployment member 235 is at least about 0.5 psi greater than the pressure of the blood of the patient 102.

The deployment member 235 can be spaced relative to the inner portion of the expandable portion 250 such that the holes direct fluid toward at least the inner portion of the modular electrode 352 in an expanded state (e.g., in an uncompressed or deployed state). For example, in a deployed state of the modular electrode 352, fluid exits the holes defined by the deployment member 235 and is directed toward an inner portion of the modular electrode 352 while an outer portion (opposite the inner portion) of the modular electrode 352 is in contact with tissue as part of diagnosis and/or as part of an ablation or other treatment. Spacing between the holes in the deployment member 235 and the inner portion of the modular electrode 352 can facilitate heat transfer between the fluid and the modular electrode 352. Additionally, or alternatively, blood can flow through the spacing between the holes in the deployment member 235 and the inner portion of the modular electrode 352. As compared to configurations in which the flow of blood away from the treatment site is impeded, the flow of blood through the spacing between the holes in the deployment member 235 and the inner portion of the modular electrode 352 can, additionally or alternatively, further improve the local heat transfer away from the outer portion of the modular electrode 352. In general, it should be appreciated that such improved local heat transfer can reduce the likelihood of unintended tissue damage during tissue treatment.

As best shown in FIG. 4, the deployment member 235 is telescoping (e.g., the deployment member 235 includes multiple concentric tubular components) such that the distalmost portion 240 of the tip section 124 can be extended and/or retracted (e.g., contracted) relative to the distal end portion 232 of the shaft 122. The telescoping feature of the deployment member 235 facilitates deployment and compression of the expandable portion 250 of the tip section 124. For example, because the deployment member 235 is mechanically coupled to the expandable portion 250 via the coupler 365 at the distalmost portion 240 of the tip section 124, axial movement of the deployment member 235 relative to the shaft 122 can exert compression and/or expansion forces on the expandable portion 250.

FIGS. 6A and 6B are schematic representations of the expandable portion 250 in a compressed state and a deployed state, respectively. Starting with the expandable portion 250 in a compressed state (FIG. 6A), proximal movement (retraction/contraction of the telescoping feature) of the deployment member 235 can pull the distal end of the expandable portion 250 in a proximal direction relative to the shaft 122 such that the expandable portion 250 expands to an uncompressed or deployed state (FIG. 6B). The deployed state of the expandable portion 250 can be used for treatment, diagnosis, or both of tissue at a treatment site. In addition, or as an alternative, distal movement (extension of the telescoping feature) of the deployment member 235 can push the distal end of the expandable portion 250 in a distal direction relative to the shaft 122 such that the expandable portion 250 collapses to a compressed state (FIG. 6A) from the deployed state (FIG. 6B). The compressed state of the expandable portion 250 can be used for retraction, delivery, or both of the tip section 124 to a treatment site. In certain implementations, the deployment member 235 can be mechanically coupled to a portion of the handle 120 (e.g., the actuation portion 246 shown in FIG. 2) such that movement of the deployment member 235 can be controlled at the handle 120.

In some embodiments, the inner portion of the modular electrode 352 along the expandable portion 250 can be closer in the compressed state than in the uncompressed state to at least a portion of a surface of the deployment member 235 and, thus, the inner portion of the modular electrode 352 can move away from at least a portion of the surface of the deployment member 235 as the expandable portion 250 is expanded from the compressed state (FIG. 6A) to the uncompressed state (FIG. 6B). It should be appreciated that various degrees of compression and expansion of the expandable portion 250 can be realized via various degrees of proximal and distal movement, respectively, of the deployment member 235. For example, the expandable portion 250 (i) can be compressed further than illustrated in FIG. 6A via additional distal movement (extension) of the deployment member 235, (ii) can be expanded further than illustrated in FIG. 6B via additional proximal movement (retraction) of the deployment member 235, and/or (iii) can be uncompressed or compressed to one or more states between the states illustrated in FIGS. 6A and 6B via axial movement of the deployment member 235 that positions the distalmost portion 240 of the tip section 124 between the positions of the distalmost portion 240 of the tip section 124 illustrated in FIGS. 6A and 6B. In some embodiments, the expandable portion 250 can be expanded until the nose portion 355, a distal portion of the active body portion 352, or both form a distal surface that is substantially normal to the deployment member 235 and can be positioned relatively flat against tissue (e.g., about an ostium of a pulmonary vein).

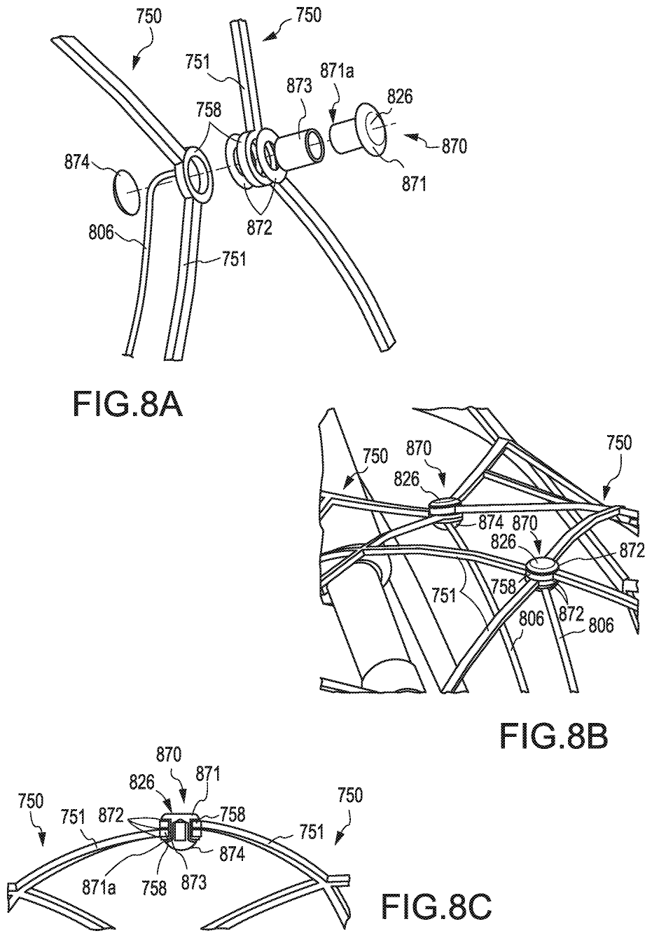

The expandable portion 250 (FIGS. 3A and 3B) is a discontinuous structure composed of a plurality of mesh electrode panels. FIGS. 7A and 7B, for example, are schematic representations of individual mesh electrode panels 750 that can be combined with other panels 750 to form the expandable portion 250. For example, as shown in FIGS. 7A and 7B, each mesh electrode panel 750 includes a plurality of struts 751, 755, and 757. All or a portion of the struts 751 form an active portion 752 of the panel 750 through which energy can be delivered to tissue. As shown, the active portion 752 of the panel is much wider than the portions of the panel 750 formed by the struts 757 and 755. In contrast, all or a portion of the struts 757 of each mesh electrode panel 750 are insulated such that electrical energy cannot be delivered to tissue through the insulated portions of the struts 757. In the illustrated embodiment, all or a portion of the struts 755 of each mesh electrode panel 750 are also insulated such that electrical energy cannot be delivered to tissue through the insulated portions of the struts 755. Additionally, or alternatively, all or a portion of the struts 755 can form part of the active portion 752 of the panel 750 through which energy can be delivered to tissue. In some embodiments, the struts 755 and/or 757 can be insulated using a PTFE sleeve or other polymer (e.g., polyimide and/or Pebax®). In other embodiments, however, the struts 755 and/or 757 may be insulated using adhesives or other suitable materials.

The panels 750 can be formed (e.g., laser cut, 3D printed, chemically etched, etc.) from a sheet or tube of material that is repeatably and reliably flexible between a compressed state and an uncompressed state. In some embodiments, the material can be at least (e.g., semi or fully) radiopaque to facilitate visualization of the material while it is within the patient 102. One example of a material that meets either or both of the above criteria is nitinol. After forming each mesh electrode panel 750 from the material, one or more surfaces of the panel 750 can be electropolished. Such electropolishing can, for example, be useful for smoothing surfaces and/or otherwise producing fine adjustments in the amount of material used to form each panel 750. Additionally, or alternatively, the material for forming each mesh electrode panel 750 can be coated with one or more of gold, tantalum, iridium oxide, or other materials. Thus, continuing with the above example, all or a portion of the active portion 752 of a mesh electrode panel 750 can optionally be coated to deliver electrical energy to tissue.

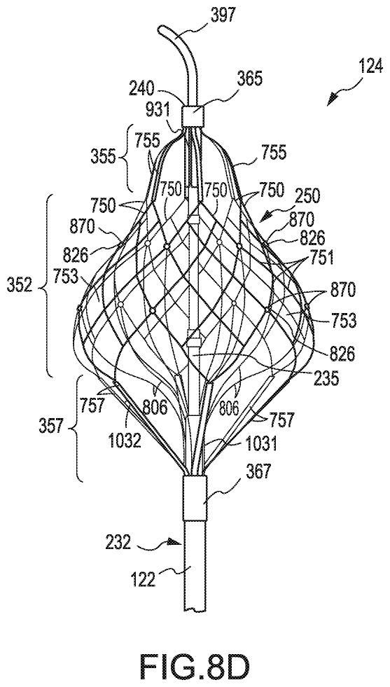

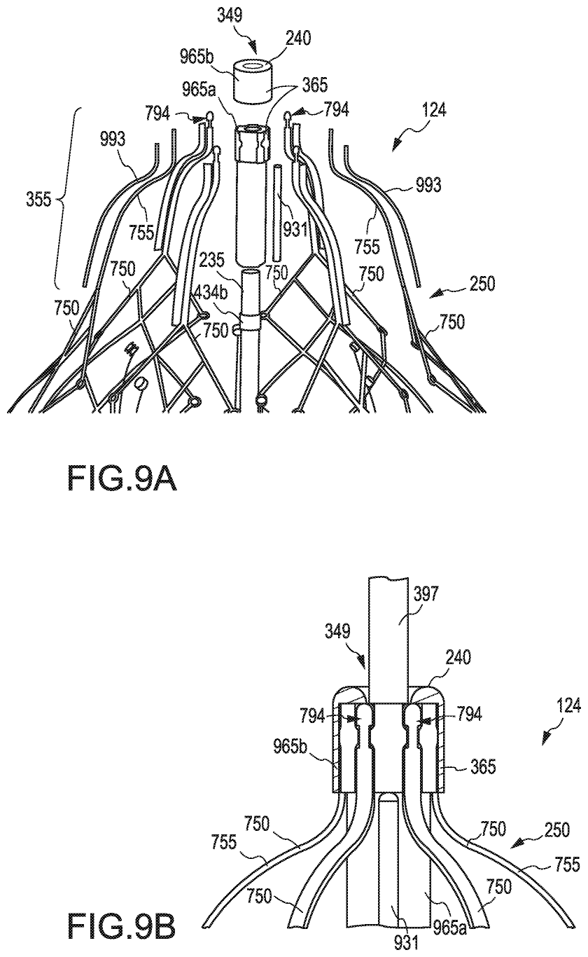

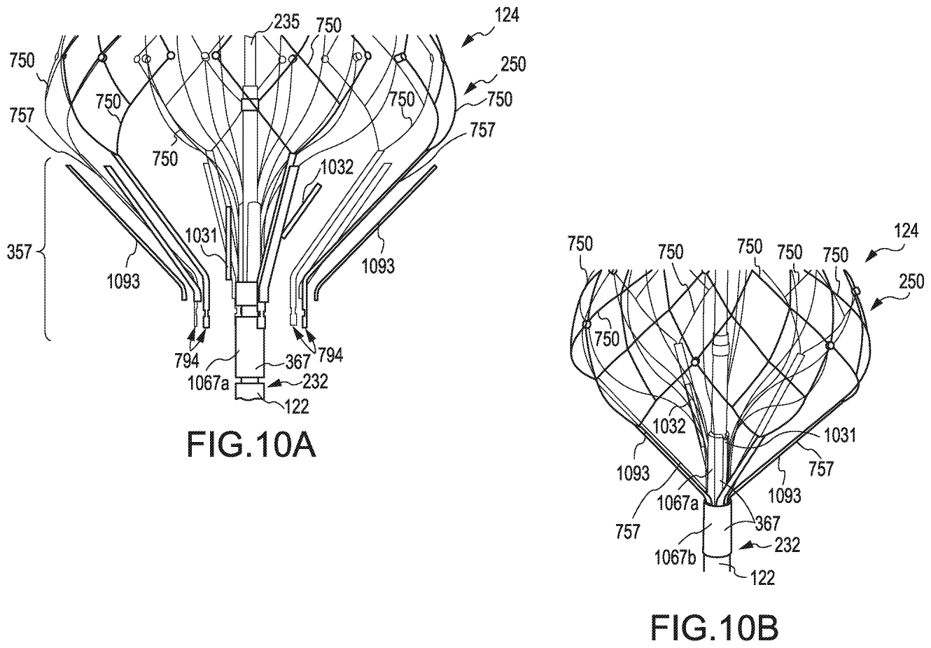

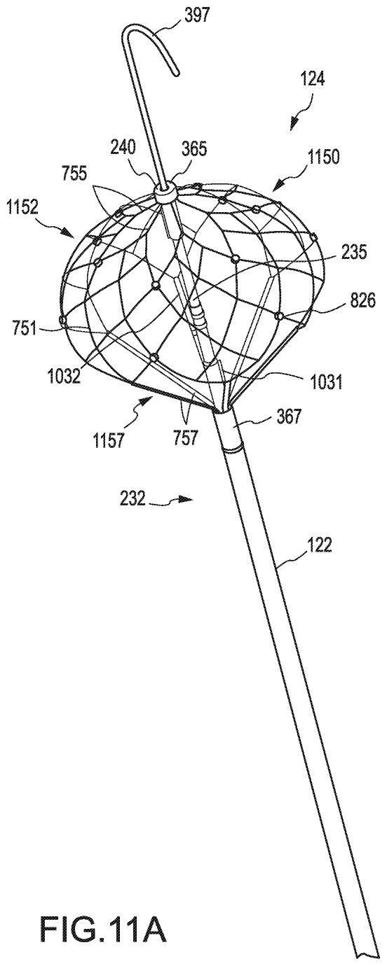

The struts 751 of each mesh electrode panel 750 can be mechanically coupled to one another to define collectively a plurality of cells 753. Accordingly, each cell 753 can be bounded by at least three struts 751 (e.g., by at least four struts 751). Also, or instead, each strut 751 can define a portion of at least one of the cells 753. In some embodiments, the cells 753 can be bounded by different numbers of struts 751, which can facilitate achieving a target distribution of current density along the expandable portion 250 (FIGS. 2-3B) when the panels 750 are mechanically coupled to one another, as described in greater detail below with respect to FIGS. 8A-8D.

Also, or instead, at least some of the struts 751 can be coupled (a) to the struts 757 to transition between a portion of the panel 750 corresponding to the modular electrode 352 of the expandable portion 250 and a portion of the panel 750 corresponding to the neck portion 357 of the expandable portion 250 and (b) to the struts 755 to transition between a portion of the panel 750 corresponding to the modular electrode 352 of the expandable portion 250 and a portion of the panel 750 corresponding to the nose portion 355 of the expandable portion 250. For example, an electrode panel 750 configured in accordance with various embodiments of the present technology can include at least one strut 757 (e.g., a single strut 757) to form at least a part of the neck portion 357 of the expandable portion 250. A proximal end (e.g., a proximal-most) portion of the strut 757 can be mechanically coupled to the distal end portion 232 of the shaft 122. A proximal end (e.g., a proximal-most) portion of a first strut 751 can be coupled to the strut 757 (e.g., to a distal end portion and/or a distalmost portion of the strut 757), and a proximal end (e.g., a proximal-most) portion of a second strut 751 can be coupled to the strut 757 (e.g., to the distal end portion and/or the distalmost portion of the strut 757). Therefore, in some embodiments, the strut 757 and the first and second struts 751 of an electrode panel 750 can form a “Y” shape at a transition between a portion of the panel 750 corresponding to the neck portion 357 of the expandable portion 250 and a portion of the panel 750 corresponding to the modular electrode 352 of the expandable portion 250.

Additionally, or alternatively, an electrode panel 750 configured in accordance with various embodiments of the present technology can include at least one strut 755 (e.g., a single strut 755) to form at least a part of the nose portion 355 (and/or at least a part of the modular electrode 352, as discussed in greater detail below with respect to FIGS. 11A-14C) of the expandable portion 250. A distal end (e.g., a distalmost) portion of the strut 755 can be mechanically coupled to the distalmost portion 240 of the tip section 124. A distal end (e.g., a distalmost) portion of a first strut 751 can be coupled to the strut 755 (e.g., to a proximal end portion and/or a proximal-most portion of the strut 755), and a distal end (e.g., a distalmost) portion of a second strut 751 can be coupled to the strut 755 (e.g., to the proximal end portion and/or a proximal-most portion of the strut 755). Therefore, in some embodiments, the strut 755 and the first and second struts 751 of an electrode panel 750 can form a “A” shape at or near a transition between a portion of the panel 750 corresponding to the nose portion 355 (and/or the modular electrode 352) of the expandable portion 250 and a portion of the panel 750 corresponding to the modular electrode 352 of the expandable portion 250.

In these and other embodiments, at least some of the cells 753 of the panel 750 are symmetric. Such symmetry can, for example, facilitate achieving the target distribution of current density along the modular electrode 352. Additionally, or alternatively, such symmetry can be useful for achieving suitable compressibility of the expandable portion 250 for delivery to a treatment site while also achieving suitable expansion of the expandable portion 250 for use at the treatment site.

In some embodiments, at least some of the cells 753 can have mirror symmetry. As used herein, a mirror symmetric shape includes a shape that is substantially symmetric about a plane intersecting the shape, with the substantial symmetry allowing for the presence or absence of an eyelet 758 on one or both sides of the plane intersecting the shape. For example, at least some of the cells 753 can have mirror symmetry about a respective mirror symmetry plane passing through the respective cell 753 and containing a center axis defined by the shaft 122 (FIGS. 1 and 2) and extending from a proximal end portion to a distal end portion of the shaft 122. Additionally, or alternatively, it should be appreciated that the overall expandable portion 250 (FIGS. 3A and 3B) of the tip section 124 can be symmetric about one or more planes including the center axis. Symmetry of the expandable portion 250 can, for example, facilitate symmetric delivery of energy to tissue at various positions about the expandable portion 250.

The mirror symmetry of at least some of the cells of the plurality of cells 753 and/or the overall expandable portion 250 can be useful, for example, for achieving the target distribution of current density. Additionally, or alternatively, symmetry can facilitate expansion and contraction of the expandable portion 250 in a predictable and repeatable manner (e.g., with little to no plastic deformation). For example, each of the cells of the plurality of cells 753 can be symmetric about its respective symmetry plane in the compressed state and in the uncompressed state of the expandable portion 250.

At least some of the plurality of cells 753 can be flexible in the axial and lateral directions such that an open framework formed by a plurality of cells 753 along the expandable portion 250 is similarly flexible when multiple panels 750 are mechanically coupled together, as described in greater detail below. For example, at least some of the plurality of cells can be substantially diamond-shaped in the uncompressed state of the expandable portion 250. As used herein, substantially diamond shaped includes shapes having a first pair of joints substantially aligned along a first axis and a second pair of joints substantially aligned along a second axis, different from the first axis (e.g., perpendicular to the first axis).

In the embodiment illustrated in FIGS. 7A and 7B, the lengths of the struts 751 decrease in a direction from a proximal region of the panel 750 (near the strut 757) to a distal region of the panel 750 (near the strut 755), which contributes to a general “pear” shape of the expandable portion 250 when multiple panels 750 are mechanically coupled to one another. In other embodiments, however, the lengths of the struts 751 can be uniform, increase, or vary non-monotonically across a mesh electrode panel 750 in the same or similar direction (from the proximal region of the panel 750 to the distal region of the panel 750) to, for example, contribute to another general shape (e.g., an “onion” shape) of the expandable portion 250. Additionally, or alternatively, a bottom section of the panel 750 comprising a subset of the struts 751 that correspond to the modular electrode 352 of the expandable portion 250 can be wider than a top section of the panel 750 comprising a different subset of the struts 751 that correspond to the modular electrode 352 of the expandable portion 250 (e.g., to contribute to the general shape of the expandable portion 750 when multiple panels 750 are mechanically coupled to one another).

In these and other embodiments, the widths of the struts 751, 755, and/or 757 can vary (e.g., across a single strut 751, 755, and/or 757, and/or across multiple struts 751, 755, and/or 757). The different widths of the struts 751, 755, and/or 757 can help provide a desired stiffness at a given position on a panel 750. For example, the widths of the struts 755 and/or 757 can be greater than the widths of the struts 751 such that the active portions 352 of the panels 750 formed by the struts 751 are more flexible than the struts 755 and/or 757. Continuing with this example, as multiple panels 750 are mechanically coupled to one another (as described in greater detail below), the modular electrode 352 of the expandable portion 250 formed from the active portions 352 of the panels 750 can be more flexible (e.g., more conformable) than the neck portion 357 formed from the struts 757 and/or the nose portion 355 formed from the struts 755. In these and other embodiments, the nose portion 355 formed from the struts 755 can be more flexible than the neck portion 357 formed from the struts 757, allowing the nose portion 355 to expand more than the neck portion 357 with respect to the deployment member 235 as the expandable portion 250 is deployed (e.g., expanded). In some embodiments, the widths of a strut 757 can vary. For example, a strut 757 can include a first portion having a first width and a second portion having a second width less than the first width to promote bending (e.g., along the second portion, at a transition between the first portion and the second portion, etc.). Additionally, or alternatively, the lengths of the struts 755 and/or 757 can be greater than the lengths of the struts 751.

As also shown in FIGS. 7A and 7B, the material removed from the sheet or tube of material can define keyed portions 794 at ends of the struts 755 and 757. As discussed in greater detail below, the keyed portions 794 of the struts 755 and 757 facilitate connecting the panels 750 to the deployment member 235 and to the distal end portion 232 of the shaft 122, respectively.

Additionally, or alternatively, the material removed from the sheet or tube of material can define eyelets 758 disposed at one end of at least some of the struts 751. The eyelets 758 can be, for example, defined at the intersection of two or more of the struts 751 and can be used to couple (e.g., mechanically couple) multiple mesh electrode panels 750 to one another. The panels 750 illustrated in FIGS. 7A and 7B each include four eyelets 758, with two of the eyelets 758 positioned on each side of an axis running from the strut 757 to the strut 755. In other embodiments, however, the panels 750 can include a lesser (e.g., one, two, or three) or greater (e.g., five or more) total number of eyelets, and/or a lesser (e.g., zero or one) or greater (e.g., three or more) number of eyelets positioned on either side of the axis running from the strut 757 to the strut 755. In these and other embodiments, the panels 750 can include eyelets 758 at other positions than illustrated in the embodiments shown in FIGS. 7A and 7B. For example, at least one of the eyelets 758 can be positioned between ends of one of the struts 751.

FIGS. 8A-8D schematically illustrate how mesh electrode panels 750 are attached to form the expandable portion 250 of the tip section 124. As shown in FIGS. 8A-8C, eyelets 758 of adjacent mesh electrode panels 750 are aligned (overlapped) and held together with a fastener 870. In the illustrated embodiment, the fastener 870 is a rivet having a primary head 871. In such implementations, the eyelets 758 of adjacent mesh electrode panels 750 can be, for example, aligned with one another such that a primary head 871 of a fastener 870 passes through the aligned eyelets 758. The primary head 871 is hollow (at least in part) such that a bottom portion 871a of the primary head 871 can be flared outward to hold the fastener 870 within the eyelets 758 and to hold the panels 750 together through force exerted on the corresponding eyelets 758 by the fastener 870. In other embodiments, a different type of fastener 870 (e.g., a two-headed rivet, a crimp, a flange screw and washer, a PEM® fastener, etc.) can be used. In some embodiments, the fastener 870 can be formed of a material (e.g., a polymer such as polyetheretherketone (PEEK)) different than the material used to form the mesh electrode panels 750. As described in greater detail below, the primary head 871 of at least some of the fasteners 870 can accommodate and/or include a sensor 826, which can be in electrical communication with the interface unit 108 (FIG. 1) and/or the generator 115 (FIG. 1) via one or more electrical leads 806 that run the length of the shaft 122 (FIGS. 1 and 2) and/or the handle 120 (FIGS. 1 and 2).

Polymer disks 872 formed of electrically insulating material (e.g., any of various different biocompatible polymers, such as polyimide) are used to separate and electrically isolate adjacent mesh electrode panels 750 from one another and/or from sensors 826 included in the fastener 870. In the illustrated embodiment, the polymer disks 872 are shown as separate disks. In other embodiments, one or more of the polymer disks 872 can form a single, integrated insulating piece. In these and other embodiments, a portion of an electrical lead 806 (e.g., a flexible printed circuit) can electrically isolate adjacent mesh electrode panels 750 from one another and/or from sensors 826 included in the fastener. In some embodiments, the portion of the electrical lead 806 can replace a polymer disk 872 (e.g., the top polymer disk 872 shown illustrated between the primary head 871 of the fastener 870 and the eyelet 758 of the top panel 750 in FIG. 8A).

Additionally, or alternatively, a grommet 873 can be disposed in the orifice of the aligned eyelets 758, between the sensor 826 and the panels 750. The grommet 873 can be formed, for example, of an electrically insulating material (e.g., any of various different biocompatible polymers). In this manner, the grommet 873 can electrically isolate the sensor 826 from the mesh electrode panels 750. Additionally, or alternatively, the grommet 873 can be formed of a pliable material to facilitate, for example, press fitting the grommet 873 and the sensor 826 through the orifice. In some embodiments, the grommet 873 can include a bottom portion (not shown) that can be flared outward (e.g., to hold the grommet 873 in place, to provide electrical isolation, etc.). Additionally, or alternatively, the grommet 873 can include a bottom flange portion (not shown). In some embodiments, the bottom flange portion can replace a polymer disk 872 (e.g., a bottommost polymer disk 872, the polymer disk 872 illustrated between the eyelets 758 in FIG. 8A, etc.) or a portion of a polymer disk 872/insulating piece. In general, the grommet 873 can reduce the likelihood that mounting the sensor 826 in the orifice will interfere with operation of the sensor 826. For example, the grommet 873 can facilitate mounting the sensor 826 to the panels 750 of the expandable portion 250 without requiring physical modification (e.g., drilling) of the sensor 826.

An encapsulant 874 can be formed over the backside of the fastener 870 and/or of the sensor 826 (e.g., to electrically insulate a portion of the sensor 826 that does not contact tissue, to electrically isolate the sensor 826 from the panels 750, and/or to electrically isolate the individual panels 750 from one another). In some embodiments, the encapsulant 874 can be an adhesive that is cured in place. In other embodiments, the encapsulant 874 can be reflowed thermoplastic or another insulator.

In some embodiments, at least one of the fasteners 870 does not accommodate or include a sensor 826. Such a fastener 870 can be formed of a material (e.g., a polymer such as PEEK) different than the material used to form the mesh electrode panels 750. In these embodiments, one or more of the polymer disks 872 (e.g., with the exception of a polymer disk 872 positioned between eyelets 758 of adjacent electrode panels 750 and used to electrically isolate the panels 750 from one another), the grommet, and/or the encapsulant 874 can be omitted from fasteners 870 that do not include a sensor 826.

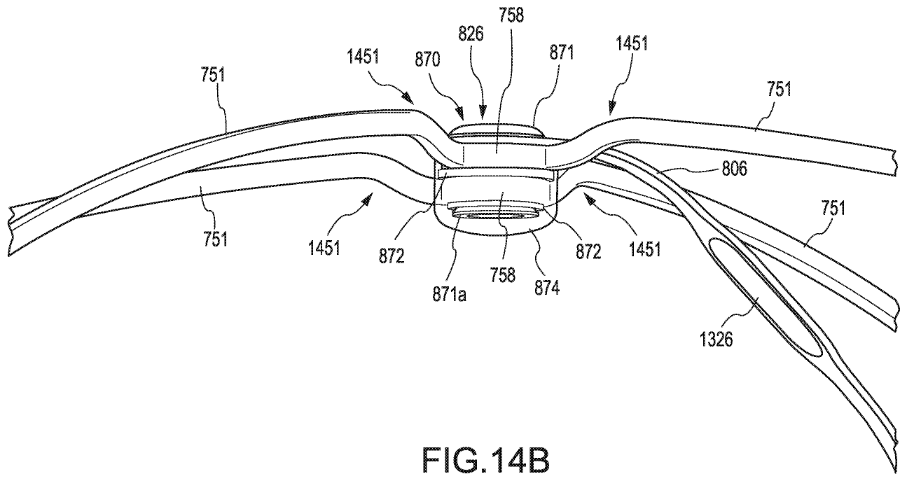

As described in greater detail below with respect to FIGS. 14A-14C, one or more of the struts 751 of at least one panel 750 may include one or more features (e.g., one or more bends) proximate one or more of the corresponding eyelets 758 in some embodiments. Such features can facilitate recessing the corresponding sensor(s) 826 relative to the exterior of the expandable portion 250 when the corresponding panels 750 are attached to one another. This can be advantageous, for example, in allowing the expandable portion 250 to pass smoothly into and out of an introducer sheath (e.g., without the corresponding sensor(s) 826 catching on a lip of the introducer sheath at one or more openings of the sheath).

Although the panels 750 are illustrated in FIGS. 7A-8D as having one or more eyelets 758 that facilitate mechanically coupling the panels 750 to one another, the panels 750 and/or the expandable portion 250 in other embodiments can include other attachment means in addition to or in lieu of the eyelets 758 for mechanically coupling the panels 750 to one another. For example, outer joints of one or more of the cells 753 along the perimeter of a panel 750 can be mechanically coupled to corresponding outer joints of one or more of the cells 753 along the perimeter of another panel 750 using a fastener (e.g., using a figure-eight non-conductive fastener that passes through a cell 753 on the perimeter of each of the panels 750, using a non-conductive fastener that weaves back and forth between the panels 750 from the struts 755 to the struts 757 (and/or vice versa) passing through one or more of the cells 753 on the perimeter of each of the panels 750, etc.). Independent of the method of coupling the panels 750 to one another, in some embodiments, insulating material (e.g., grommet, sleeve, adhesive, dipcast, heat shrink, reflowed thermoplastic, or another appropriate material) may be applied to the struts 751 and/or the eyelets 758 to provide additional electrical insulation between adjacent mesh electrode panels 750.

Referring now to FIG. 8D, as multiple (e.g., two, three, four, five, six, or more (e.g., seven to twelve)) mesh electrode panels 750 are mechanically coupled to one another, the panels 750 collectively form a closed shape to define the expandable portion 250. In the illustrated embodiment, six mesh electrode panels 750 form a pear-shaped expandable portion 250. In particular, all or a portion of the struts 751 of the panels 750 form the modular electrode 352 of the expandable portion 250. Additionally, as described in greater detail below, the struts 755 of the panels 750 form the nose portion 355 of the expandable portion 250, and the struts 757 of the panels 750 form the neck portion 357 of the expandable portion 250.

As shown, a center section of the expandable portion 250 corresponding to the modular electrode 352 is much wider than a first section corresponding to the neck portion 357 of the expandable portion 250 and/or a second section corresponding to the nose portion 355 (and/or a portion of the modular electrode 352, as discussed in greater detail below with respect to FIGS. 11A-14C) of the expandable portion 250. Additionally, or alternatively, the expandable portion 250 includes a greater number of cells 753 about an equator of the expandable portion 250 than about the first and/or second sections.

As shown, multiple cells 753 of the expandable portion 250 are bounded by at least four struts (e.g., by struts 751, struts 757, and/or struts 755). Several of the cells 753 are each bounded by struts (e.g., by struts 751, struts 757, and/or struts 755) belonging to different (e.g., adjacent) electrode panels 750. For example, a cell 753 of the expandable portion 250 at the nose portion 355 is bounded by a strut 755 and at least one strut 751 of a first panel 750, as well as by a strut 755 and at least one strut 751 of a second (e.g., adjacent) panel 750. Similarly, a cell 753 of the expandable portion 250 at the neck portion 357 is bounded by a strut 757 and at least one strut 751 of a first panel 750, as well as by a strut 757 and at least one strut 751 of a second (e.g., adjacent) panel 750. As still another example, a cell 753 of the expandable portion 250 at the modular electrode 352 is bounded by at least one strut 751 of a first panel 750 and by at least one strut 751 of a second (e.g., adjacent) panel 750.

In the uncompressed state, the struts 751, the eyelets 758 (shown filled with fasteners 870 in FIG. 8D), and the cells 753 formed by the struts 751 together form an open framework having a conductive surface along at least a portion of the modular electrode 352 of the expandable portion 250. For example, the open framework formed by the struts 751, the eyelets 758, and the cells 753 can have greater than about 50 percent open area along the outer portion of the modular electrode 352 when the expandable portion 250 is in the uncompressed state. Continuing with this example, in the uncompressed state, the combined open area of the cells 753 can be greater than the combined area of the struts 751 and the eyelets 758 along the outer portion of the modular electrode 352. Further, or instead, at least some of the cells 753 can have a larger area in the uncompressed state of the expandable portion 250 than in the compressed state of the expandable portion 250.

As discussed above, the expandable portion 250 can be expanded (e.g., deployed) and compressed via proximal and axial movement, respectively, of the deployment member 235. To facilitate movement of the expandable portion 250 from a compressed state to a deployed state (and vice versa), the struts 751 of each of the panels 750 can be flexible relative to one another. For example, a maximum radial dimension (alternatively referred to herein as a lateral dimension) of the modular electrode 352 can increase by at least a factor of two as the coupled struts 751 move relative to one another to transition the expandable portion 250 from a fully compressed state to a fully uncompressed state, in the absence of external force. Additionally, or alternatively, the struts 751 can be movable relative to one another such that a maximum radial dimension of the expandable portion 250 in the uncompressed state is at least about 20 percent greater than a maximum radial dimension of the shaft 122 (e.g., greater than a maximum radial dimension of the distal end portion 232 of the shaft 122). For example, the expandable portion 250 in the uncompressed state illustrated in FIG. 8D has an outer diameter of greater than about 20 mm and less than about 40 mm (e.g., a diameter between 28 mm and 30 mm, or about 29 mm), while the shaft 122 has an outer diameter greater than about 1.5 mm and less than about 7 mm (e.g., a diameter between 2 mm and 3.5 mm, or about 2.7 mm). This ratio of increase in size is achieved through the use of the open framework of cells 753 formed by the struts 751, which makes use of less material than would otherwise be required for a solid shape of the same size.

In some embodiments, the expandable portion 250 can assume its expanded (e.g., pear) shape in the absence of external force. For example, when the expandable portion 250 is not mechanically coupled (e.g., is not tethered) to the distalmost portion 240 of the tip section 124, to the distal end portion 232 of the shaft 122, and/or to another portion of the catheter; and/or when no other compression forces are acting on the expandable portion 250, the expandable portion 250 can assume its deployed shape. In some embodiments, the expanded or deployed shape can have a diameter greater than a maximum diameter of the catheter shaft 122.

It should be appreciated that the open area of the expandable portion 250 can facilitate the flow of fluid and blood through expandable portion 250 during treatment. In other words, the inner portion of the modular electrode 352 can be in fluid communication with the outer portion of the modular electrode 352 through the plurality of cells 753 such that, in use, fluid, blood, or a combination thereof can move through the plurality of cells 753 to cool the modular electrode 352 and tissue in the vicinity of the modular electrode 352. As compared to electrodes that impede the flow of blood, the open area of the expandable portion 250 can reduce the likelihood of local heating of blood at the treatment site as energy is delivered to the tissue. Furthermore, as compared to electrodes that impede the flow of blood, the open area of the expanded portion 250 can reduce the likelihood of blood coagulation or clot formation, thereby reducing the likelihood of thromboembolism. It should also be appreciated that the delivery of fluid to the inner portion of the modular electrode 352 can augment the cooling that occurs through the flow of only blood through the open area.

As discussed above, the struts 755 of the panels 750 together form the nose portion 355 of the expandable portion 250, and the struts 757 of the panels 750 together form the neck portion 357 of the expandable portion 250. FIGS. 9A-10B illustrate how the mesh electrode panels 750 of the expandable portion 250 are attached to the deployment member 235 (shown in FIGS. 9A and 9B) and to the distal end portion 232 of the shaft 122 (shown in FIGS. 10A and 10B).

Referring first to FIGS. 9A and 9B, the coupler 365 at the distalmost portion 240 of the tip section 124 couples the struts 755 to the deployment member 235 (FIG. 9A). In some embodiments, the coupler 367 is a machined hard insulator, such as a PEEK coupler. In the illustrated embodiment, the coupler 365 includes a first portion 965a and a second portion 965b. The first portion 965a includes recessed portions configured to receive corresponding keyed portions 794 of the struts 755. The recessed portions of the first portion 965a of the coupler 365 prevent axial movement (within machined tolerances) of the keyed portions 794 of the struts 755 relative to the distalmost portion 240 of the tip section 124. The second portion 965b of the coupler 365 fits over the first portion 965a to retain the keyed portions 794 of the struts 755 within the recessed portions of the first portion 965a.

In other embodiments, the struts 755 can be coupled to the distalmost portion 240 of the tip section 124 using other types of couplers 365. For example, the ends of the struts 755 can include an aperture configured to receive a centering pin or rivet of the coupler 365. The coupler 365 in these embodiments can include a corresponding second portion configured to receive and retain the centering pin or rivet to align the ends of the struts 755 to the second portion and retain the ends of the struts 755 in place. Still other examples of couplers 365 within the scope of the present technology include heat stakes, crimps, ultrasonic welds, or reflows to retain the ends of the struts 755 in place.

As discussed above, in the illustrated embodiment, all or a portion of the struts 755 are insulated. As best shown in FIG. 9A, the struts 755 include an insulator 993 (e.g., a polyethylene terephthalate (PET) or PTFE sleeve) that covers a majority of the struts 755. In some embodiments, the insulators 993 terminate before the keyed portions 794 of the struts 755. In other embodiments, the insulators 993 extend to and/or coat all or a portion of the keyed portions 794. Additionally, or alternatively, the coupler 365 may include insulating materials to prevent electrical connection between the mesh electrode panels 750.

Referring now to FIGS. 10A and 10B, the coupler 367 couples the struts 757 of the mesh electrode panels 750 to the distal end portion 232 of the shaft 122. In some embodiments, the coupler 367 is a machined hard insulator, such as a PEEK coupler.

In the illustrated embodiment, the coupler 367 includes a first portion 1067a (FIG. 10A) and a second portion 1067b (FIG. 10B). The first portion 1067a includes recessed portions configured to receive corresponding keyed portions 794 of the struts 757. The recessed portions of the first portion 1067a of the coupler 367 prevent axial movement (within machined tolerances) of the keyed portions 794 of the struts 755 relative to the distal end portion of the shaft 122. The second portion 1067b of the coupler 367 fits over the first portion 1067a to retain the keyed portions 794 of the struts 757 within the recessed portions of the first portion 1067a.

In other embodiments, the struts 757 can be coupled to the distal end portion 232 of the shaft 122 using other types of couplers 367. For example, the ends of the struts 757 can include an aperture configured to receive a centering pin or rivet of the coupler 367. The coupler 367 in these embodiments can include a corresponding second portion configured to receive and retain the centering pin or rivet to align the ends of the struts 757 to the second portion and retain the ends of the struts 757 in place. Still other examples of couplers 367 within the scope of the present technology include heat stakes, crimps, ultrasonic welds, or reflows to retain the ends of the struts 757 in place.

As discussed above, all or a portion of the struts 757 are insulated. As best shown in FIG. 10A, the struts 757 include an insulator 1093 (e.g., a PTFE sleeve) that covers a majority of the struts 757. In some embodiments, the insulators 1093 terminate before the keyed portions 794 of the struts 757. In other embodiments the insulators 1093 extend to and/or coat all or a portion of the keyed portions 794. Additionally, or alternatively, at least a portion of the coupler 367 may include insulating materials to prevent electrical connection between the mesh electrode panels 750.

The coupler 367 can include electrical contacts electrically coupled to the generator 115 (FIG. 1) via one or more of the electrical leads or wires 148 (FIG. 1) and/or other conductive paths extending from the generator 115 along the length of the shaft 122. In these and other embodiments, when the struts 757 are secured within the coupler 367, each of the struts 757 can be directly or indirectly electrically coupled to the generator 115 (FIG. 1) via one of more of the electrical contacts of the coupler 367 and/or via one or more of the electrical leads or wires 148 and/or other conductive paths extending from the generator 115 along the length of the shaft 122. In this manner, as described in greater detail below, electrical energy provided by the generator 115 can be separately delivered to the struts 751 of individual mesh electrode panels 750 of the expandable portion 250 via the struts 757 of the panels 750, where the electrical energy can be delivered to tissue of the patient 102 via a corresponding section of the modular electrode 352.

Referring again to FIGS. 8A-8D, sensors 826 can be mounted along the modular electrode 352 of the expandable portion 250 at all or a subset of the locations where adjacent mesh electrode panels 750 are mechanically coupled to one another by eyelets 758 and the fasteners 870. In general, the sensors 826 can be positioned along one or both of the inner portion and the outer portion of the modular electrode 352.

Each sensor 826 can be electrically insulated from the panels 750 and mounted on and/or within the fasteners 870. For example, each sensor 826 can be mounted to the fasteners 870 using a compliant adhesive (e.g., epoxy or a room temperature vulcanized (RTV) silicone), any of various different mechanical retaining features (e.g., tabs) between the sensor 826 and the fastener, and/or molding or overmolding of the sensor 826 to the fastener 870. Additionally, or alternatively, the sensors 826 can extend through a portion of the fasteners 870 and/or the eyelets 758 of the panels 750. Such positioning of the sensors 826 through a portion of the fasteners 870 can facilitate forming a robust mechanical connection between the sensors 826 and the fasteners 870. Additionally, or alternatively, positioning the sensors 826 through a portion of the fasteners can facilitate measuring conditions along the outer portion and the inner portion of the modular electrode 352.

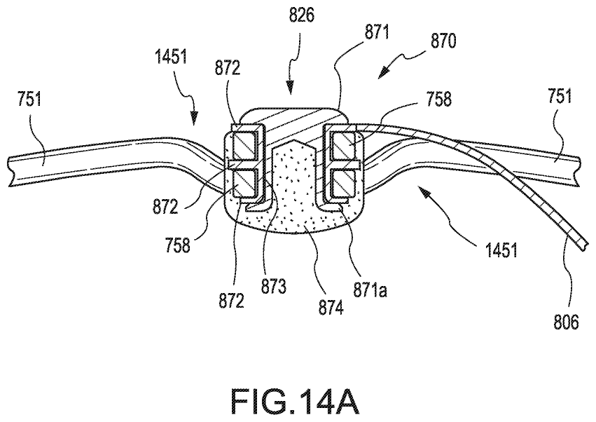

Electrical leads 806 extend from each sensor 826, within or along the interior of the expandable portion 250 and into the shaft 122 (FIG. 2). The electrical leads 806 may comprise wires (e.g., insulated wires) or printed circuits (e.g., flexible printed circuits) or a combination thereof. The electrical leads 806 are in electrical communication with the interface unit 108 (FIG. 1) and/or the generator 115 (FIG. 1) such that each sensor 826 can send electrical signals to and receive electrical signals (e.g., electrical energy) from the interface unit 108 and/or the generator 115 during use. As discussed in greater detail below with respect to FIG. 13, one or more additional sensors (not shown in FIGS. 1-10B) can be formed by and/or positioned on one or more of the electrical leads 806 such that the one or more additional sensors remain within the interior of the expandable portion 250 and do not contact tissue when the expandable portion 250 is in contact with tissue.

The sensors 826 can be substantially uniformly spaced from one another (e.g., in a circumferential direction and/or in an axial direction) along the modular electrode 352 of the expandable portion 250 when the expandable portion 250 is in an uncompressed state. Such substantially uniform distribution of the sensors 826 can, for example, facilitate determining a shape (e.g., an extent of expansion and/or deformation) and/or temperature profile of all or portions of the modular electrode 352 during use. For example, the sensors 826 can be electrically isolated from the modular electrode 352, with the sensors 826 (acting as surface electrodes) passively detecting electrical activity of tissue in proximity to each respective sensor 826 without interference from the modular electrode 352. At least some of the sensors 826 can be at least partially disposed along an outer portion of the expandable portion 250 with the expandable portion 250 between one or more internal electrodes (e.g., the ring electrode 434a, the ring electrode 434b, and/or one or more additional sensors formed by and/or positioned on one or more of the electrical leads 806) and at least a portion of each respective one of the sensors 826 along the outer portion. Additionally, or alternatively, at least some of the sensors 826 can extend through the modular electrode 352. In these embodiments, one or more of the sensors 826 can be insulated along the inner portion of the expandable portion 250 and/or exposed along the outer portion of the expandable portion 250. Also, or instead, at least some of the sensors 826 can be at least partially disposed and/or exposed along an inner portion of the expandable portion 250. In such implementations, each sensor 826 can be in proximity to tissue without touching tissue as the modular electrode 352 touches tissue.

Each sensor 826 can act as an electrode (e.g., a surface electrode) to detect electrical activity of the heart in an area local to the sensor 826. In some embodiments, one or more of the sensors 826 can be coated with platinum black or iridium oxide (e.g., to reduce impedance or noise). Additionally, or alternatively, each sensor 826 can include a temperature measurement device (e.g., a thermocouple or a thermistor). For example, a sensor 826 can comprise a flexible printed circuit, a temperature measurement device secured between portions of the flexible printed circuit, and a termination pad opposite the temperature measurement device. Continuing with this example, the sensor 826 can be mounted on a fastener 870 with a thermistor disposed along the outer portion of the expandable portion 250 and a termination pad disposed along the inner portion of the expandable portion 250. In certain instances, the thermistor can be disposed along the outer portion to provide an accurate indication of tissue temperature. A thermally conductive adhesive or other conductive material can be disposed over the thermistor to secure the thermistor to the flexible printed circuit. In these and other embodiments, one or more of the sensors 826 can include an ultrasound transducer, an optical fiber, and/or other types of image sensors. As another example, a sensor 826 can include a flexible printed circuit with two or more electrodes, one of which is disposed along the outer portion of the expandable portion 250. As yet another example, a sensor 826 can include a thermocouple formed at the junction of two metals within the sensor 826 (e.g., within a flexible printed circuit comprising, for example, constantan and copper traces) or at the point of electrical connection between the sensor 826 and an electrical lead 826.

In some implementations, each sensor 826 can be formed of and/or include a radiopaque material. The radiopacity of the sensors 826 can, for example, facilitate visualization (e.g., using fluoroscopy) of the sensors 826 during use. Examples of radiopaque material that can form and/or be added to the sensor 826 include: platinum, platinum iridium, gold, radiopaque ink, and combinations thereof. The radiopaque material can be formed and/or added in any pattern that may facilitate visualization of the radiopaque material such as, for example, a dot and/or a ring.

In certain implementations, each sensor 826 can form part of an electrode set useful for detecting contact between each sensor 826 and tissue. For example, electrical energy (e.g., current) can be driven through each sensor 826 and another electrode or a plurality of other electrodes (e.g., any one or more of the various different electrodes described herein) and a change in a measured signal (e.g., voltage or impedance) can be indicative of the presence of tissue. Because the position of the tip section 124 is known, the detection of contact through respective measured signals at the sensors 826 can be useful for determining a shape of the anatomic structure in which the tip section 124 is disposed and/or tissue engagement/contact with the tip section 124 during the course of a medical procedure. Additionally, or alternatively, measured signals at the sensors 826 can be useful for determining the position of an introducer sheath relative to the tip section 124, for example by detecting an increase in a measured signal (e.g., voltage or impedance) when the sensor is covered by the sheath, thereby indicating that the sheath is at least partially covering the tip section 124.

In use, each sensor 826 can, further or instead, act as an electrode to detect electrical activity in an area of the heart local to the respective sensor 826, with the detected electrical activity forming a basis for an electrogram associated with the respective sensor 826 and, further or instead, can provide lesion or other feedback. The sensors 826 can be arranged such that electrical activity detected by each sensor 826 can form the basis of unipolar electrograms and/or bipolar electrograms. For example, in embodiments in which one or more additional sensors are formed by and/or positioned on one or more electrical leads 806, the sensors 826 can cooperate with the additional sensors to form one or more bipolar electrograms. Additionally, or alternatively, in embodiments in which the sensors 826 include a flexible printed circuit comprising two or more electrodes, the two or more electrodes of a sensor 826 can cooperate to form one or more bipolar electrograms. Additionally, or alternatively, the sensors 826 can cooperate with a center electrode (e.g., a ring electrode 434a and/or 434b (FIG. 4) on the deployment member 235) to provide near-unipolar electrograms, as described in greater detail below. For example, electrical activity detected (e.g., passively detected) by the center electrode and the sensors 826 (acting as surface electrodes) can form the basis of respective electrograms associated with each unique pairing of the center electrode and the sensors 826. As a more specific example, in implementations in which there are six sensors 826, the center electrode can form six electrode pairs with the sensors 826 which, in turn, form the basis for six respective electrograms. An electrogram formed by electrical signals received from each respective electrode pair (e.g., the center electrode and a respective one of the sensors 826) can be generated through any of various different methods. In general, an electrogram associated with a respective electrode pair can be based on a difference between the signals from the electrodes in the pair and, thus more specifically, can be based on a difference between an electrical signal received from the center electrode and an electrical signal received from a respective one of the sensors 826. Electrograms can be filtered or otherwise further processed to reduce noise and/or to emphasize cardiac electrical activity, for example. It should be appreciated that the sensors 826 and a center electrode can cooperate to provide near-unipolar electrograms in addition, or as an alternative, to any one or more of the various different methods of determining contact, shape, force, and impedance described herein, each of which may include further or alternative cooperation between the sensors 826 and a center electrode.

The plurality of sensors 826 can be used to detect deformation of the expandable portion 250 (e.g., of the modular electrode 352). For example, electrical signals can be driven between the ring electrodes 434a and/or 434b on the deployment member 235 and each of the plurality of sensors 826 according to any of the methods described herein. Additionally, or alternatively, electrical signals can be driven between one of the sensors 826 and another of the sensors 826, or between one of the sensors 826 and an additional sensor formed by and/or positioned on one of the electrical leads 806. Additionally, or alternatively, electrical signals can be driven between two or more electrodes of one of the sensors 826. Measured electrical signals generated between (i) at least one of the sensors 826 and another of the sensors 826, (ii) at least one of the sensors 826 and the ring electrode(s) 434a and/or 434b, (iii) at least one of the sensors 826 and at least one additional sensor formed by and/or positioned on the electrical leads 806, and/or (iv) two or more electrodes of at least one of the sensors 826 can be received at the processing unit 110 (FIG. 1).

Based at least in part on the measured electrical signals, the shape (e.g., the extent of expansion and/or deformation) of the expandable portion 250 can be detected. For example, as the expandable portion 250 is deformed, one or more of the sensors 826 can be brought into contact with the deployment member 235. It should be appreciated that a certain amount of force is required to deform the expandable portion 250 by an amount sufficient to bring the one or more sensors 826 into contact with the deployment member 235 at least while the expandable portion 250 is in a fully deployed (uncompressed) state. As used herein, this force can be considered a threshold at least in the sense that forces below this threshold are insufficient to bring the one or more sensors 826 close to the deployment member 235 and, therefore, are not detected as contact between the one or more sensors 826 and the deployment member 235.

In some embodiments, a shaft electrode (e.g., a ring electrode on the shaft (not shown)) can be mounted to the distal end portion 232 of the shaft 122 near the neck portion 357 (e.g., on or proximate the coupler 367). The shaft electrode can be used to measure electrograms in accordance with various methods described herein. Additionally, or alternatively, electrical energy (e.g., current) can be driven through the shaft electrode and one or more other electrodes (e.g., any one or more of the various different electrodes described herein), and a change in a measured signal (e.g., voltage or impedance) can be indicative of the presence of an introducer sheath covering the shaft electrode. Continuing with this example, measured signals from the shaft electrode can be used in combination with measured signals from the sensors 826 to determine a location of the introducer sheath relative to the tip section 124 and/or the distal end portion 232 of the shaft 122.

Referring to FIG. 8D-10B, the tip section 124 can further include one or more location coil sensors (e.g., magnetic coil sensors). For example, the coupler 365 and/or the coupler 367 can include one or more slots or notches to house and/or retain one or more location coil sensors 931 (FIGS. 8D-9B) and one or more location coil sensors 1031 (FIGS. 8D, 10A, and 10B), respectively. Additionally, or alternatively, the tip section 124 can include location coil sensors 1032 (FIGS. 8D, 10A, and 10B) mounted on one or more of the struts 755 and/or 757.

In some embodiments, the location coil sensors 931, 1031, and/or 1032 are magnetic coil sensors configured to emit a magnetic field while other coils (e.g., external to the patient 102, others of the coil sensors 931, 1031, and/or 1032, etc.) can be used to measure the resultant magnetic field. Additionally, or alternatively, coils external to the patient 102 can be configured to emit a magnetic field. In these and other embodiments, the location coil sensors 931, 1031, and/or 1032 can be configured to transmit and/or receive signals indicating information relating to three to six degrees of freedom. For example, the location coil sensors 931, 1031, and/or 1032 can transmit and/or receive signals indicating positional information of the coil sensors 931, 1031, and/or 1032 in three-dimensional space (e.g., signals indicating x, y, and z positional coordinates relative to a defined origin, such as an external reference frame and/or relative to one or more of the location coil sensors 931, 1031, and/or 1032). Additionally, or alternatively, the location coil sensors 931, 1031, and/or 1032 can transmit and/or receive signals indicating pitch, yaw, and/or roll information. Therefore, the location coil sensors 931, 1031, and/or 1032 can be used to resolve the location of the tip section 124 (e.g., within the patient 102) relative to a defined origin and/or can be used to computationally determine the shape and/or orientation (e.g., pose) of the expandable portion 250. Additionally, or alternatively, the location coil sensors 931, 1031, and/or 1032 can be used (i) to determine a distance between the coil sensors 931 and the coil sensors 1031, and/or (ii) to determine a distance and/or angle between the coil sensors 931, 1031, and/or 1032. In turn, the determined distances and/or angles can be used to determine and/or estimate a shape (e.g., an extent of expansion and/or deformation) of the expandable portion 250.

Referring to FIG. 8D, various components of the tip section 124 can be used, alone or in combination, to determine the position, shape (e.g., level of expansion and/or deformation), and/or pose of the tip section 124 (e.g., of the expandable portion 250). For example, location and/or orientation information can be provided by the location coil sensors 931, 1031, and/or 1032, as discussed above. Additionally, or alternatively, fluoroscopic visualization (e.g., X-Ray, CT, etc.) can be used to determine the position, shape, and/or pose of the tip section 124. For example, in some embodiments, at least a portion of the tip section 124 is radiopaque, with the expandable portion 250 observable through the use of fluoroscopy or other similar visualization techniques. In some embodiments, the expandable portion 250 of the tip section 124 can be radiopaque such that fluoroscopy can provide an indication of the deformation and/or partial deformation of the expandable portion 250 and, therefore, provide an indication of whether the expandable portion 250 is in contact with tissue. Additionally, or alternatively, the shaft 122, the deployment member 235, the coupler 367, the coupler 365, and/or one or more of the sensors 826 can be composed of and/or coated with radiopaque materials and thus be visible using fluoroscopy or other visualization techniques.

As a specific example, a portion of the deployment member 235 encompassed by the expandable portion 250 of the tip section 124 can include three concentric tubes. Each of the concentric tubes can include a radiopaque ring, and all three rings can be fluoroscopically distinct (e.g., separated) when the deployment member 235 is fully extended distally (e.g., when the expandable portion 250 is in a fully compressed state). As the deployment member 235 is retracted (e.g., as the distalmost portion 240 of the tip section 124 is moved proximally), distal concentric tubes of the deployment member 235 slide within more proximal concentric tubes of the deployment member 235 such that the radiopaque rings on the distal concentric tubes become overlaid during fluoroscopy within the more proximal concentric tubes. As such, the extent of deployment of the expandable portion 250 can be determined based at least in part on the relative position of radiopaque rings on the deployment member 235. The portion of the deployment member 235 encompassed by the expandable portion 250 of the tip section 124 can include a greater (e.g., four or more) or lesser (e.g., two) number of concentric tubes in other embodiments, and/or can include a different number (e.g., two or more) number of radiopaque rings per concentric tube.

Additionally, or alternatively, the coupler 367 can include a radiopaque ring. In some embodiments, the radiopaque ring on the coupler 367 can be visibly distinct (e.g., via size and/or pattern) from the radiopaque elements of other components of the catheter 104 (e.g., from the radiopaque rings on the deployment member 235). Thus, the radiopaque ring on the coupler 367 can provide orientation (e.g., pose) information of the tip section 124 during fluoroscopic visualization.

In some embodiments, the shape (e.g., the extent of deployment, deformation, etc.) of the expandable portion 250 can be predicted based on the position of the deployment member 235. A displacement measuring device (potentiometer, encoder, or other devices well known in the art) in the handle 120 can be used to measure a displacement of the deployment member 235. The measured displacement can be used by the processing unit 110 (FIG. 1) to determine a shape of the expandable portion 250 for display on the graphical user interface 109 (FIG. 1).

In these and still other embodiments, electrical measurements captured by all or a subset of the sensors 826 can be used to determine the shape of the expandable portion 250. For example, impedance detected by an electrode pair (e.g., a pair of the sensors 826, a sensor 826 and the ring electrode 434a (FIG. 4), a sensor 826 and the ring electrode 434b (FIG. 4), etc.) can be detected (e.g., as a signal received by the processing unit 110 (FIG. 1)) when an electrical signal is driven through the electrode pair. The impedance detected for various electrode pairs can be compared to one another and relative distances between the members of each electrode pair determined. For example, if the sensors 826 are identical, each sensor 826 can be driven as part of a respective electrode pair including the ring electrode 434a and/or the ring electrode 434b of the deployment member 235. For each such electrode pair, the measured impedance between the electrode pair can be indicative of relative distance between the particular sensor 826 and the ring electrode 434a and/or 434b forming the respective electrode pair. In implementations in which the deployment member 235 is stationary while electrical signals are driven through the electrode pairs, the relative distance between each sensor 826 and the deployment member 235 can be further indicative of relative distance between each sensor 826 and each of the other sensors 826. In general, driven electrode pairs with lower measured impedance are closer to one another than those driven electrode pairs with higher measured impedance. In certain instances, electrodes associated with the modular electrode 352 (e.g., one or more of the sensors 826) that are not being driven can be measured to determine additional information regarding the position of the driven current pair.

The measurements received by the processing unit 110 and associated with the driven current pairs alone, or in combination with the measurements at the sensors 826 that are not being driven, can be fit to a model and/or compared to a look-up table to determine displacement of the expandable portion 250 of the tip section 124. For example, the determined displacement of the expandable portion 250 can include displacement in at least one of an axial direction or a lateral (radial) direction. It should be appreciated that, because of the spatial separation of the current pairs in three dimensions, the determined displacement of the expandable portion 250 can be in more than one direction (e.g., an axial direction, a lateral direction, and combinations thereof). Additionally, or alternatively, the determined displacement of the expandable portion 250 can correspond to a three-dimensional shape of the expandable portion 250 of the tip section 124. Thus, the determined displacement of the expandable portion 250 can be used, for example, to determine the shape of the expandable portion 250. Further, or instead, signals measured by ultrasound transducers, optical fibers, and/or other types of image sensors included in the sensors 826 and/or disposed on the deployment member 235 can be used to determine displacement (and, therefore, the shape) of the expandable portion 250.

In embodiments where the axial force-displacement and/or the lateral force-displacement response of the expandable portion 250 can be reproducible for a given deployed state, the amount of force applied to the expandable portion 250 of the tip section 124 in the axial and/or lateral direction can be reliably determined based on respective displacement of the expandable portion 250 in the given deployed state. Accordingly, the determined displacement of the expandable portion 250 can be used to determine the amount and direction of force applied to the expandable portion 250. In particular, the processing unit 110 can determine force applied to the expandable portion 250 based on the determined displacement of the expandable portion 250. For example, using a lookup table, a curve fit, or other predetermined relationship, the processing unit 110 can determine the direction and magnitude of force applied to the expandable portion 250 based on the magnitude and direction of the displacement of the expandable portion 250, as determined according to any one or more of the methods of determining displacement described herein. It should be appreciated, therefore, that the reproducible relationship between force and displacement along the expandable portion 250, coupled with the ability to determine displacement using the sensors 826 disposed along the modular electrode 352, can facilitate determining whether an appropriate amount of force is being applied during an ablation treatment and, additionally or alternatively, can facilitate determining appropriate energy and/or cooling dosing for lesion formation.

The detection and/or observation of the position, shape, and/or orientation of the tip section 124 can, for example, provide improved certainty that the expandable portion 250 is engaging tissue and/or that an intended treatment is, in fact, being provided to tissue. It should be appreciated that improved certainty of positioning of the modular electrode 352 with respect to tissue can improve the likelihood that energy is applied to tissue at the correct location within the patient and/or can reduce the likelihood of inducing stenosis in a pulmonary vein and/or gaps in a lesion pattern about the ostium of the pulmonary vein.

In some embodiments, the graphical user interface 109 (FIG. 1) can be used to display various information collected by the tip section 124 of the catheter 104. For example, the graphical user interface 109 can be used to display the catheter 104 with an icon representing the location, orientation, and/or shape of the tip section 124 and the shaft 122 on a mapping system (e.g., within a model of an anatomical structure of the patient 102). For example, based on the determined displacement of the expandable portion 250 of the tip section 124, the processing unit 110 (FIG. 1) can send an indication of the shape of the expandable portion 250 to the graphical user interface 109. Such an indication of the shape of the expandable portion 250 can include, for example, a graphical representation of the shape of the expandable portion 250 corresponding to the determined deformation. In these and other embodiments, the graphical user interface 109 can be used to display treatment location (e.g., lesion locations). Additionally, or alternatively, the graphical user interface 109 can be used to display other information corresponding to the tip section 124 of the catheter 104. For example, the graphical user interface 109 can be used to display voltage and/or temperature measurements captured by one or more of the sensors 826. As a specific example, the graphical user interface 109 can be used to display a representation of at least one of the electrograms measured by one or more of the sensors 826, a center electrode, and/or one or more additional sensors formed by and/or positioned on one or more of the electrical leads 806, and/or other information (e.g., a voltage map associated with the electrograms) corresponding to the tip section 124 of the catheter 104. As another example, the graphical user interface 109 can be used to display an electro-anatomical map based at least in part on the electrograms and/or on the determined shape and/or location of the tip section 124.

FIGS. 11A-15 illustrate tip sections 124 configured in accordance with various other embodiments of the present technology. The tip sections 124 illustrated in FIGS. 11A-15 are similar to the tip section 124 illustrated in FIGS. 2-10B. Therefore, similar reference numbers are used to indicate similar elements across FIGS. 2-15, but the individual components may not be identical. The tip sections 124 illustrated in FIGS. 11A-15 differ from the tip section 124 illustrated in FIGS. 2-10B in that the struts 751, 755, and 757 of individual mesh electrode panels 750 are sized (and the struts 755 and 757 attached to the couplers 365 and 367, respectively) to contribute to a general “onion” shape and/or a general “pumpkin” shape of expandable portions 1150 and 1550 rather than the general “pear” shape of the expandable portion 250 of the tip section 124 illustrated in FIGS. 2-10B.

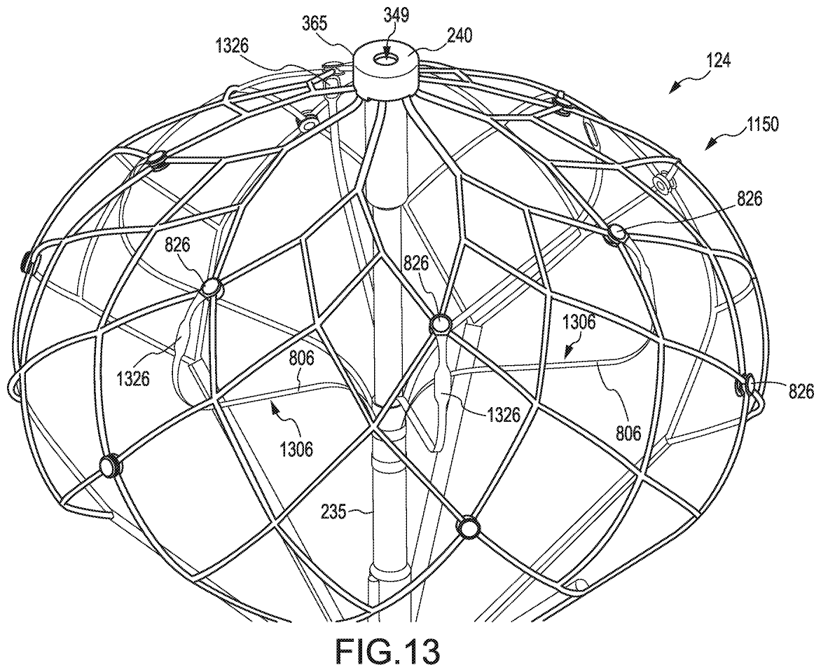

FIGS. 11A and 11B illustrate an “onion”-shaped expandable portion 1150 of a tip section 124 in a deployed state. FIGS. 12A and 12B are a top view and a top perspective view of a distalmost portion 240 of the expandable portion 1150, respectively. As best shown in FIGS. 11A and 11B, the expandable portion 1150 of the illustrated tip section 124 includes a neck portion 1157 and an active body portion 1152 (referred to hereinafter as “modular electrode 1152”). Notably, the expandable portion 1150 does not include an accentuated nose portion that is similar to the nose portion 355 of the expandable portion 250 illustrated in FIGS. 2-10B. Instead, referring to FIGS. 11A-12B together, a distal portion of the modular electrode 1152 (at least when the expandable portion 1150 is in a fully deployed state) can form a distal surface that is substantially normal to the deployment member 235 and that can be positioned relatively flat against tissue (e.g., about an ostium of a pulmonary vein). Thus, the struts 755 of the mesh electrode panels of the expandable portion 1150 can contribute to the modular electrode 1152. As such, all or a portion of the struts 755 of the expandable portion 1150 can be used in some embodiments to abut tissue (e.g., about an ostium of a pulmonary vein) and/or to deliver energy to the tissue. Additionally, or alternatively, all or a portion of the struts 755 of the expandable portion 1150 can be insulated such that the insulated portions of the struts 755 can abut tissue, but not be used to deliver energy to the tissue.

FIG. 13 illustrates a side perspective view of the expandable portion 1150 of the tip section 124. As shown, sensors 826 are distributed about the expandable portion 1150 and are electrically coupled to electrical leads 806 consistent with the discussion above with respect to FIGS. 2-10B. One or more of the electrical leads 806 illustrated in FIG. 13 include one or more additional sensors 1326 that are formed by and/or are positioned on the electrical leads 806 in such a manner that the additional sensor(s) 1326 remain within the interior of the expandable portion 1150 and do not contact tissue when the expandable portion 1150 is in contact with tissue. As discussed in greater detail above, the additional sensors 1326 can be used, for example, to form one or more electrograms and/or to measure electrical signals (e.g., voltage or impedance) to determine the shape (e.g., the extent of deployment, deformation, etc.) of the expandable portion 1150, etc. The additional sensors can be coated with platinum black, iridium oxide, or gold (e.g., to reduce electrical impedance or noise, and/or to increase thermal conductivity).

In order to allow deployment and compression of the expandable portion 1150, a service loop 1306 can be included in some embodiments for one or more of the electrical leads 806. The service loops 1306 can be maintained in place within the expandable portion 1150 by various means including (i) wrapping or spiraling the electrical leads 806 around the deployment member 235 or (ii) joining two or more electrical leads 806 in a “Y” shape that at least partially engages with the deployment member 235.

FIGS. 14A-14C illustrate how mesh electrode panels 750 can be attached to form the expandable portion 1150 of the tip section 124, consistent with the discussion of FIGS. 8A-8C above. For example, eyelets 758 of adjacent mesh electrode panels 750 can be aligned (overlapped) and held together with fasteners 870. Polymer disks 872 formed of electrically insulating material can be used to separate and electrically isolate adjacent mesh electrode panels 750 from one another and/or from sensors 826 included in the fastener 870. Additionally, or alternatively, a grommet 873 (FIG. 14A) can be disposed in the orifice of the aligned eyelets 758, between the sensor 826 and the panels 750 (e.g., to electrically isolate the sensor 826 from the panels 750).

An encapsulant 874 can be formed over the backside of the fastener 870 and/or of the sensor 826 (e.g., to electrically insulate a portion of the sensor 826 that does not contact tissue, to electrically isolate the sensor 826 from the panels 750, and/or to electrically isolate the individual panels 750 from one another). In some embodiments, the encapsulant 874 can be an adhesive that is cured in place. In other embodiments, the encapsulant 874 can be reflowed thermoplastic or another insulator.

The struts 751 of the panels 750 illustrated in FIGS. 14A-14C include one or more features or bends 1450 proximate the corresponding eyelets 758. When these panels 750 are attached to one another, the bends 1450 in the struts 751 can facilitate recessing the sensor 826 included in the fastener 870 sensor(s) 826 relative to the exterior of the expandable portion 1150. As discussed above, this can be advantageous, for example, in allowing the expandable portion 1150 to pass smoothly into and out of an introducer sheath (e.g., without the corresponding sensor(s) 826 catching on a lip of the introducer sheath at one or more openings of the sheath).

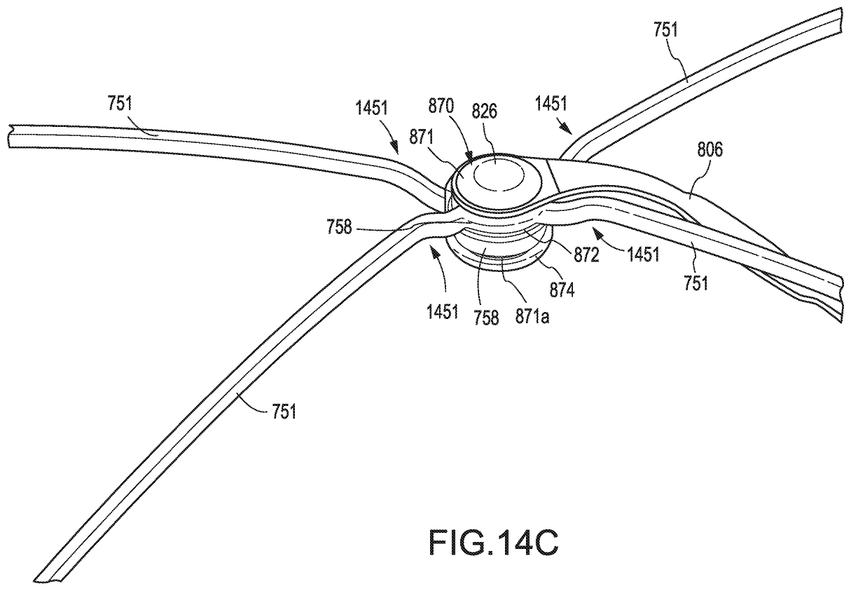

FIG. 15 illustrates an expandable portion 1550 of a tip section 124 in a deployed state. The expandable portion 1550 is similar to the expandable portion 1150 illustrated in FIGS. 11A-14C except that the struts 755 of the panels 750 are attached to the distalmost part of the coupler 365. As such, the struts 755 emerge from the coupler 365 in a distal direction to form an inverted nose portion 1555. The inverted nose portion 1555 includes a distalmost portion 1540 of the expandable portion 1550 and prevents the coupler 365 (e.g., the distalmost portion 240 of the tip section 124) from making contact with tissue when the expandable portion 1550 is in a fully (or substantially fully) deployed state. This can be advantageous, for example, to deliver energy to tissue using a distal face or surface of the expandable portion 250 without interference from the coupler 365. Thus, in some embodiments, all or a portion of the struts 755 of the expandable portion 1550 can be used to abut and/or deliver energy to tissue. Additionally, or alternatively, all or a portion of the struts 755 of the expandable portion 1550 can be insulated such that the insulated portions of the struts 755 can abut tissue but are not used to deliver energy to the tissue.

2. Associated Methods

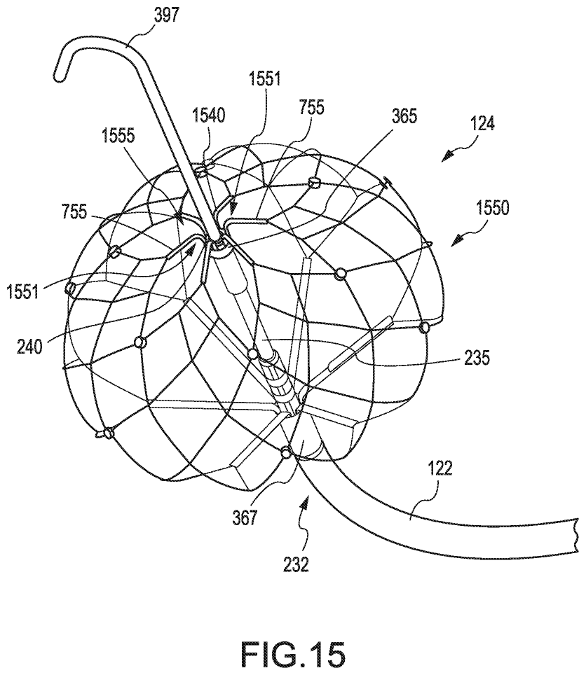

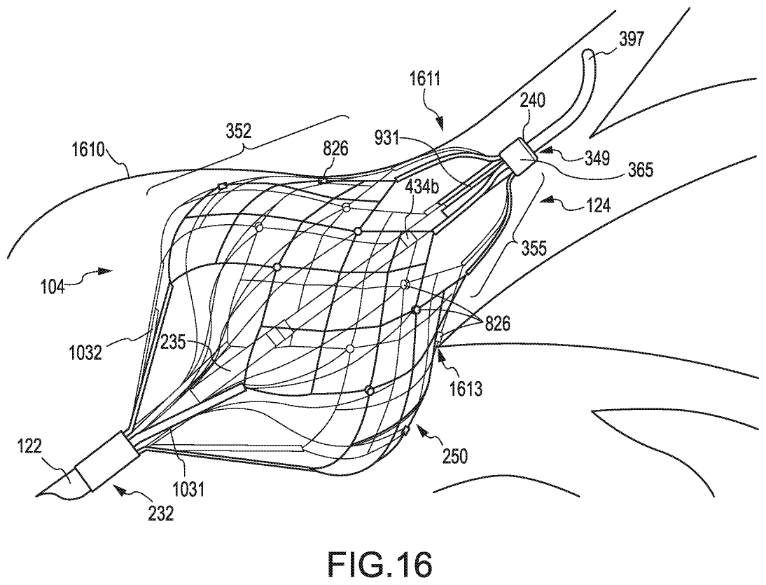

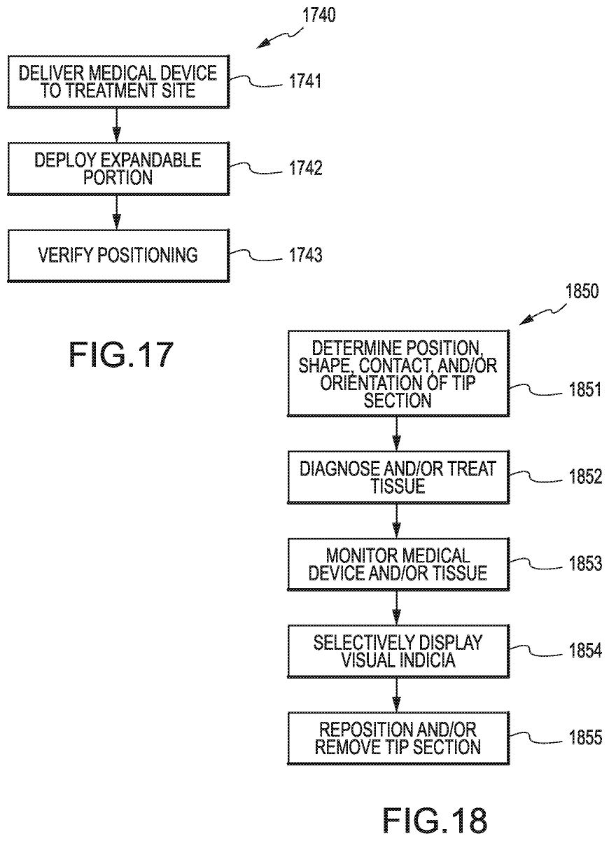

FIG. 16 is a schematic representation of the tip section 124 of FIGS. 2-10B positioned at a treatment site within an anatomical structure of a patient (in this case, proximate an ostium 1613 of a pulmonary vein 1611 in the left atrium of the patient's heart 1610) in accordance with various embodiments of the present technology. For the sake of clarity and explanation, FIGS. 17 and 18 are discussed below in conjunction with FIG. 16. A person skilled in the art will readily recognize, however, that all or a portion of the methods described in FIGS. 17 and 18 can be applied using tip sections 124 configured in accordance with various other embodiments of the present technology, such as tips sections 124 having expandable portions within modular electrodes similar to the expandable portions 1150 and/or 1550 with modular electrodes 1152 illustrated in FIGS. 11A-15. Additionally, a person skilled in the art will readily recognize that all or a portion of the methods described in FIGS. 17 and 18 can be applied in contexts other than pulmonary vein isolation procedures, such as in any of various medical procedures performed on a hollow anatomical structure of a patient, and, more specifically, in procedures for diagnosing, stimulating, electrically isolating, or treating tissue within and/or proximal the anatomical structure.

FIG. 17 is a flow diagram illustrating a method 1740 for positioning a tip section of a catheter at a treatment site within an anatomical structure of a patient in accordance with various embodiments of the present technology. All or a subset of the steps of the method 1740 can be executed by various components or devices of a medical system, such as the system 100 illustrated in FIG. 1 or other suitable systems. For example, all or a subset of the steps of the method 1740 can be executed by (i) components or devices of the interface unit 108, (ii) components or devices of the medical device 104, and/or (iv) the mapping system 112, the recording system 113, the fluid pump 114, and/or the generator 115. Additionally, or alternatively, all or a subset of the steps of the method 1740 can be executed by a user (e.g., operator, physician, etc.) of the system 100. Furthermore, any one or more of the steps of the method 1740 can be executed in accordance with the discussion above.

Referring to FIGS. 16 and 17 together, the method 1740 begins at block 1741 by delivering the tip section 124 of the catheter 104 to a treatment site within an anatomical structure of a patient. For example, the tip section 124 can be inserted into a patient in a compressed state and delivered into a patient's heart 1610 via a vein in the patient's leg or arm. In some embodiments, the tip section 124 can be navigated to a pulmonary vein 1611 in the patient's heart (e.g., to an ostium 1613 of a pulmonary vein 1611 in the left atrium of the patient's heart 1610) using an introducer sheath (e.g., a steerable introducer sheath such as an Abbott Agilis) and/or a guidewire 397. In these and other embodiments, fluoroscopy and/or other visualization techniques can be used to navigate the tip section 124 to the treatment site. In these and still other embodiments, positioning information provided by location coil sensors 931, 1031, and/or 1032 and/or the sensors 826 distributed about the deformable portion 250 of the tip section 124 can be used to navigate the tip section 124 to the treatment site.

At block 1742, the expandable portion 250 of the tip section 124 is deployed at the treatment site. For example, the expandable portion of the tip section 124 can be deployed by retracting the deployment member 235 of the tip section 124 relative to the distal end portion 232 of the shaft. In some embodiments, deployment can include retracting the deployment member 235 using an actuation portion 246 (FIG. 2) on the handle 120 (FIG. 2) of the catheter 104.

To deploy the expandable portion 250 of the tip section 124, the nose portion 355 of the expandable portion 250 can be advanced in a compressed state into the pulmonary vein 1611 and subsequently expanded to a deployed state corresponding to the size of the pulmonary vein 1611. As the expandable portion 250 expands, the nose portion 355 can engage the walls of the pulmonary vein 1611 to center the nose portion 355 within the pulmonary vein 1611. Additionally, or alternatively, the pear shape of the expandable portion 250 enables the walls of the pulmonary vein 1611 to push at least a portion of the modular electrode 352 of the expandable portion 250 out of the ostium of the pulmonary vein 1611 into the left atrium of the heart 1610, thereby preventing portions of the modular electrode 352 from engaging tissue within the pulmonary vein 1611. In this manner, the tip section 124 of the catheter 104 can be deployed to a size corresponding to the size of the pulmonary vein 1611 while ensuring that only insulated portions of the expandable portion 250 are beyond the ostium of the pulmonary vein 1611 and that the active portion (i.e., the modular electrode 352) of the expandable portion 250 is properly positioned against tissue in the left atrium of the heart 1610 about the ostium 1613 of the pulmonary vein 1611 (as opposed to against tissue within the pulmonary vein 1611). As such, the likelihood of stenosis of the pulmonary vein 1611 as a result of treatment is reduced.

In other embodiments, to deploy the expandable portion 250 of the tip section 124, the expandable portion 250 can be expanded to a deployed state before advancing at least the nose portion 355 of the expandable portion 250 into the pulmonary vein 1611. For example, the expandable portion 250 can be expanded to a fully deployed state or to an approximate size of the pulmonary vein 1611. The nose portion 355 of the expandable portion 250 is then advanced toward and/or into the pulmonary vein 1611. If the nose portion 355 is successfully advanced into the pulmonary vein 1611, the expandable portion 250 can be further expanded in some embodiments until the nose portion 355 engages the walls of the pulmonary vein 1611 and is centered within the pulmonary vein 1611. On the other hand, if the nose portion 355 cannot be successfully advanced into the pulmonary vein 1611, the expandable portion 250 can be compressed via distal movement of the deployment member 235 relative to the distal end portion 232 of the shaft 122 until the nose portion 355 is successfully advanced into the pulmonary vein 1611.

In these and still other embodiments, the expandable portion of the tip section 124 can be expanded to a deployed state until the nose portion and/or a distal portion of the modular electrode form a distal surface that is substantially normal to the deployment member 235. The distal surface can then be positioned relatively flat against tissue about the ostium 1613 of the pulmonary vein 1611 (e.g., with the coupler 365 and/or at least a portion of the struts 755 positioned within the pulmonary vein 1611).

At block 1743, the method 1740 verifies that the tip section 124 is properly positioned at the treatment site. In particular, the method 1740 verifies that (e.g., a distal surface of) the modular electrode 352 engages tissue in the left atrium of the heart 1610 about the ostium 1613 of the pulmonary vein 1611. Additionally, or alternatively, the method 1740 verifies that the tip section 124 (e.g., the nose portion 355) is centered within the pulmonary vein 1611. In these and other embodiments, the method 1740 verifies that the struts 755 and not the coupler 365 are properly positioned against tissue.

In some embodiments, to verify placement of the tip section 124 in the pulmonary vein 1611, the guidewire 397 can be further advanced into the pulmonary vein 1611 and/or contrast dye can be injected into the pulmonary vein 1611 (e.g., out of an opening of the lumen 349 of the deployment member 235) to verify proper placement. In these and other embodiments, the position, shape (e.g., deformation and/or level of expansion), and/or orientation of the tip section 124 can be determined in accordance with any one or more of the various different methods described herein (e.g., fluoroscopic visualization; position and/or orientation information provided by the location coil sensors 931, 1031, and/or 1032; impedance measurements using the sensors 826 and/or the ring electrodes 434a and/or 434b of the deployment member 235; etc.) to verify positioning of the tip section 124 at the treatment site. In these and still other embodiments, to verify proper positioning of the tip section 124 at the treatment site, the extent of contact between all or a portion of the modular electrode 352 and tissue at the treatment site can be determined in accordance with any one or more of the various different methods described herein.

Although the steps of the method 1740 are discussed and illustrated in a particular order, the method 1740 illustrated in FIG. 17 is not so limited. In other embodiments, the method 1740 can be performed in a different order. In these and other embodiments, any of the steps of the method 1740 can be performed before, during, and/or after any of the other steps of the method 1740. Moreover, a person of ordinary skill in the relevant art will recognize that the illustrated method can be altered and still remain within these and other embodiments of the present technology. For example, one or more steps of the method 1740 illustrated in FIG. 17 can be omitted and/or repeated in some embodiments.

FIG. 18 is a flow diagram illustrating a method 1850 for diagnosing and/or treating tissue at a treatment site within an anatomical structure of a patient in accordance with various embodiments of the present technology. For example, FIG. 18 is a flow diagram illustrating a method 1850 for diagnosing and/or treating tissue within the left atrium of a patient's heart 1610 (FIG. 16) about the ostium 1613 (FIG. 16) of a pulmonary vein 1611 (FIG. 16) to electrically isolate the pulmonary vein 1611 from the patient's heart 1610. All or a subset of the steps of the method 1850 can be executed by various components or devices of a medical system, such as the system 100 illustrated in FIG. 1 or other suitable systems. For example, all or a subset of the steps of the method 1850 can be executed by (i) components or devices of the interface unit 108, (ii) components or devices of the medical device 104, and/or (iv) the mapping system 112, the recording system 113, the fluid pump 114, and/or the generator 115. Additionally, or alternatively, all or a subset of the steps of the method 1850 can be performed by a user (e.g., operator, physician, etc.) of the system 100. Furthermore, any one or more of the steps of the method 1850 can be executed in accordance with the discussion above.

Referring to FIGS. 16 and 18 together, the method 1850 begins at block 1851 by determining a position, shape, contact, and/or orientation of a tip section 124 of a catheter 104 at the treatment site. For example, the position, shape, contact, and/or orientation of the tip section 124 can be determined in accordance with any one or more of the various methods described herein and/or in a similar manner as described above with respect to block 1243 of the method 1240 (FIG. 12). In some embodiments, the size and/or effective surface area of the expandable portion 250 of the tip section 124 corresponding to the current level of deployment (expansion) of the expandable portion 250 can be determined. In these and other embodiments, which portions (e.g., which mesh electrode panels 750 (FIGS. 7A and 7B), which portions of an individual panel, etc.) of the modular electrode 352 that are currently contacting tissue at the ostium 1613 of the pulmonary vein 1611 can be determined. In these and still other embodiments, the effective surface area of the modular electrode 352 (e.g., of the entire modular electrode 352, of each individual panel, etc.) in contact with tissue about the treatment site can be determined.

At block 1852, the method 1850 can diagnose and/or treat tissue at the treatment site. In some embodiments, tissue is diagnosed by measuring characteristics of the tissue in accordance with any one or more of the various methods described herein. For example, electrical signals of the tissue can be measured using one or more of the sensors 826 distributed about the modular electrode 352, using the modular electrode 352 itself, using the ring electrodes 434a and/or 434b of the deployment member 235, and/or using one or more of the additional sensors 1326 (FIG. 13) formed by and/or positioned on one or more of the electrical leads 806. Based at least in part on the measured electrical signals, one or more electrograms and/or electro-anatomical maps corresponding to tissue in contact with and/or proximate to the sensors 826, the modular electrode 352, the ring electrode 434a and/or 434b, and/or the one or more of the additional sensors 1326 formed by and/or positioned on one or more of the electrical leads 806 can be generated. In this manner, the method 1850 (i) can identify tissue exhibiting abnormal electrical behavior that may be contributing to a condition of the patient 102 (e.g., to atrial fibrillation) and/or (ii) can track the electrical behavior of tissue as the tissue is treated. In these and other embodiments, the tissue can be diagnosed by measuring electrical activation (e.g., using one or more of the sensors 826) and/or by pacing the heart 1610 of the patient 102. In these and still other embodiments, the tissue can be diagnosed by determining thickness of tissue (e.g., based on an anatomical location of the tissue and/or on other measurements captured by the catheter 104).

In some embodiments, energy (e.g., electrical energy) can be delivered to select panels of the modular electrode 352 to treat tissue at the treatment site. In turn, the select panels of the modular electrode 352 can deliver energy to the tissue. For example, radiofrequency (RF) energy can be delivered to one or more panels of the modular electrode 352 using the generator 115 (FIG. 1). As a more specific example, the method 1850 can deliver between approximately 1 ampere and 4 amperes (e.g., between about 2 amperes and 3 amperes, or about 2.6 amperes) at approximately 500 kHz (e.g., 400 kHz and 600 kHz) to each panel together and/or separately for about 4 seconds (e.g., between 2 seconds and 9 seconds total, between 3 seconds and 5 seconds per panel, etc.). In some embodiments, parameters of RF energy delivered to tissue through the modular electrode 352 can be adjusted (e.g., altered) based on a variety of factors, as discussed in greater detail below.

As another example, pulsed field ablation (e.g., irreversible electroporation) and/or another form of energy can be delivered to one or more of the panels of the modular electrode using the generator 115 to treat tissue at the treatment site. As a more specific example, the method 1850 can deliver between approximately 18 amperes and 60 amperes (e.g., between about 20 amperes and 26 amperes, or about 24 amperes) to each panel together and/or separately with bi-phasic pulses of approximately 1 microsecond (e.g., 0.5 microseconds to 5 microseconds) that are repeated approximately every 1 ms (e.g., 0.5 ms to 10 ms) for a total of about 3 seconds (e.g., 1.5 seconds to 10 seconds).

Additionally, or alternatively, the method 1850 can deliver various forms of pulse trains of energy to one or more of the panels of the modular electrode using the generator 115. For example, the method 1850 can deliver a train of tightly (e.g., temporally) spaced pulses of energy followed by a suspension period during which no energy is delivered. At the end of the suspension period, the method 1850 can deliver another train of tightly spaced pulses of energy followed by another suspension period. The method 1850 can repeat this cycle as needed. In still other embodiments, the method 1850 can vary the amount of current delivered during different pulses (e.g., of a pulse train). In some embodiments, parameters of pulsed field and/or other energy delivered to tissue through the modular electrode 352 can be adjusted (e.g., altered) based on a variety of factors discussed in greater detail below.

In these and other embodiments, to treat tissue at the treatment site, channels, relays, and/or transistors of the generator 115 (FIG. 1) can be used to separately and/or concurrently drive the mesh electrode panels of the expandable portion 250. In some embodiments, all or a subset of the panels of the modular electrode 352 can be driven in a monopolar electrode configuration (e.g., between the panels and one or more return electrodes 118 (FIG. 1) external to the patient 102 (FIG. 1)). In embodiments having multiple return electrodes 118, the method 1850 can balance current returned via each of the electrodes 118. More information regarding current balancing between return electrodes can be found, for example, in U.S. patent application Ser. No. 16/493,288, assigned to Affera, Inc., which is incorporated herein by reference in its entirety. Alternatively, the panels can be driven in a bipolar electrode configuration. For example, the method 1850 can deliver energy between adjacent and/or separated (e.g., opposite) panels, and/or the method 1850 can deliver energy between one or more mesh electrode panels and a center electrode (e.g., a ring electrode on the deployment member and/or another electrode of the tip section 124).

In some embodiments, the method 1850 includes driving each of the mesh electrode panels together. In these and other embodiments, the method 1850 includes driving individual panels separately from one another. For example, assuming the expandable portion 250 includes six panels, the method 1850 can drive the panels in the following order: (i) the first panel; (ii) the second panel; (iii) the third panel; (iv) the fourth panel; (v) the fifth panel; and (vi) the sixth panel. In some embodiments, time division is used to separately drive the panels of the expandable portion 250.

In these and still other embodiments, the method 1850 can include simultaneously driving the panels in various subgroupings. For example, assuming the expandable portion 250 includes six panels, the method 1850 can drive the panels in the following order: (i) a first panel together with a second panel adjacent the first panel; (ii) the second panel together with a third panel adjacent the second panel; (iii) the third panel together with a fourth panel adjacent the third panel; (iv) the fourth panel together with a fifth panel adjacent the fourth panel; (v) the fifth panel together with a sixth panel adjacent the fifth panel; and (vi) the sixth panel together with the first panel adjacent the sixth panel. In these and still other embodiments, the method 1850 can drive other groupings of the panels (e.g., groups of two panels that are opposite one another on the expandable portion 250, groups of two panels separated by an adjacent panel, groups of three or more panels, groups of every other panel, etc.). Other groupings of the panels are of course possible and within the scope of the present technology. In some embodiments, time division is used to separately drive the groupings of the panels.

When driving the panels of the expandable portion 250 separately to treat tissue, the method 1850 may perform only one instance of energy delivery per panel. In other embodiments, the method 1850 may perform multiple instances of (e.g., lesser) energy delivery per panel. For example, the method 1850 can sequentially energize individual panels a second time after the method 1850 sequentially energizes the individual panels a first time. In some embodiments, multiple instances of energy delivery per panel can allow a panel to cool after the method 1850 drives the panel and before the method 1850 subsequently drives the panel again

Selectively driving the panels of the modular electrode offers several advantages over conventional pulmonary vein isolation catheters. For example, instead of delivering a large amount of energy (e.g., 900 J) into a patient 102 all at once, the method 1850 can deliver the same total amount of energy into the patient 102 over time by delivering a smaller amount of energy per panel (e.g., 150 J per panel in the case of an expandable portion 250 comprising six panels) while also maintaining a same or similar current density across the expandable portion 250 (because the effective surface area of a subset of the panels is lesser than the effective surface area of the entire expandable portion 250). Additionally, or alternatively, the extent of tissue contact, the extent of panel deployment/deformation, and other characteristics of tissue at the treatment site and/or of the individual panels can vary across the expandable portion 250. Therefore, selectively driving the panels of the modular electrode 352 according to tissue characteristics and other factors local to a panel offers greater granularity and control over energy delivered to tissue at the treatment site through each portion of the modular electrode 352, as described in greater detail below.

In some embodiments, energy delivery can be synchronized with the refractory period of ventricular activation. For example, the method 1850 can trigger energy delivery to the modular electrode 352 at a predetermined time delay relative to when the method 1850 detects ventricular activation via an electrode (e.g., the modular electrode 352, one or more of the sensors 826, etc.) and/or when pacing the ventricle.

In these and other embodiments, the method 1850 can tailor energy delivered to the modular electrode 352 based on a variety of factors. In some embodiments, the position, shape, contact, and/or orientation information of a tip section 124 determined at block 1851 and/or the characteristics of tissue determined during diagnosis of the tissue at block 1852 can be used to tailor energy delivered to the tissue via the modular electrode 352. For example, energy delivery can be tailored based on anatomical location of tissue. As a specific example, the method 1850 can determine that a first panel is contacting a thinner (e.g., posterior) section of tissue at the treatment site and that a second panel is contacting a thicker (e.g., anterior) section of tissue at the treatment site. Accordingly, the method 1850 in some embodiments can deliver less energy via the first panel of the modular electrode 352 contacting the thinner section of tissue than energy delivered via the second panel to the thicker section of tissue.

In these and other embodiments, because current density at a given point along the modular electrode 352 is a function of the effective surface area at the given point along the modular electrode 352, energy delivered to the panels of the modular electrode can be tailored based on the shape (e.g., level of expansion and/or deformation) of the expandable portion 250 to maintain a target current density of energy delivered to tissue through each panel of the modular electrode 352 in contact with tissue. For example, a pulmonary vein having a smaller diameter will require less energy overall to treat tissue about the ostium of the smaller pulmonary vein than a pulmonary vein having a larger diameter. Continuing with this example, the expandable portion 250, when properly positioned in the smaller pulmonary vein, will be less deployed (and therefore have a less effective surface area) than the expandable portion 250 when properly positioned in the larger pulmonary vein. Therefore, the method 1850 can deliver less energy to the panels of the modular electrode 352 when the modular electrode 352 is positioned within the smaller pulmonary vein and can deliver more energy to the panels of the modular electrode 352 when the modular electrode 352 is positioned within the larger pulmonary vein. As additional examples, a first panel of the modular electrode 352 can be more deformed (e.g., via contact with tissue) and/or have a lesser amount of the first panel in contact with tissue than a second panel of the modular electrode 352. In either or both of these scenarios, the method 1850 can determine that the first panel has a smaller effective surface area than the second panel and can therefore deliver less energy to the first panels than the to the second panel to maintain a target current density of energy delivered to tissue through each panel of the expandable portion 250.

At block 1853, various parameters of the medical device and/or the tissue are monitored during diagnosis, treatment, or both of the tissue. For example, during RF energy delivery, one or more of the sensors 826 can measure temperature of the tissue and/or portions of the modular electrode 352. In these embodiments, the amount and/or temperature of irrigation fluid delivered to the treatment site can be varied based at least in part on the temperature measurements. For example, the method 1850 can increase the flow rate and/or decrease the temperature of irrigation fluid delivered to the modular electrode 352 and/or to tissue at the treatment site as the temperature of tissue and/or the temperature of portions of the modular electrode 352 increase. Additionally, or alternatively, the energy delivered to a panel of the modular electrode 352 can be adjusted based at least in part on temperature measurements captured by one or more sensors 826 corresponding to and/or proximate the panel. For example, the method 1850 can decrease or terminate energy delivered to a panel when temperature measurements corresponding to the panel meet or exceed a threshold temperature. In some embodiments, the method 1850 can wait to resume delivering energy to the panel until temperature measurements corresponding to the panel drop below the threshold temperature. In this manner, the method 1850 can reduce the likelihood of clotting or charring treated tissue. In some embodiments, the method 1850 can continue to deliver energy to tissue about the ostium via other panels of the modular electrode 352 when temperature measurements corresponding to the other panels are at or below the threshold temperature or another temperature. Alternatively, the method 1850 can decrease or terminate energy delivered to other panels of the modular electrode 352 (e.g., to all of the other panels; to a subset of the other panels, such as adjacent panels; etc.) in addition to decreasing or terminating energy delivered to the panel whose corresponding temperature measurements meet or exceed the threshold temperature.

In these and other embodiments, temperature measurements captured by one or more of the sensors 826 can be used for other purposes. For example, during RF or pulsed field energy delivery, an increase in temperature captured by a sensor 826 can be indicative of contact between the corresponding panel(s) and tissue at the treatment site. Thus, the temperature measurements captured by one or more of the sensors 826 can be used to determine which portions of the modular electrode 352 are contacting tissue and, also or instead, which portions of the modular electrode 352 are unexpectedly contacting tissue (e.g., in the event that the tip section 124 has unexpectedly moved relative to the treatment site). In this manner, the method 1850 can reduce the likelihood of treating tissue other than target tissue at the treatment site. Stated another way, the method 1850 can increase the likelihood of accurately treating target tissue at the treatment site. In these and still other embodiments, the temperature measurements can be used to determine lesion characteristics and accordingly adjust energy delivery.

In some embodiments, electrical activity of target tissue at the treatment site can be monitored. For example, impedance can be monitored between (i) various pairs of the sensors 826, (ii) various one of the sensors 826 and a ring electrode 434a and/or 434b, (iii) various electrodes of one or more sensors 826, and/or (iv) various sensors and one or more additional sensors 1326 (FIG. 13) formed by and/or positioned on one or more of the electrical leads 806. Impedance measurements can be used to determine the shape of the expandable portion 250 (e.g., to determine that the expandable portion 250 is unexpectedly deformed) and/or as feedback of lesion characteristics (as impedance changes when tissue is treated). As another example, electrograms provided by one or more of the sensors 826 corresponding to the tissue at the treatment site can be monitored. Such feedback can be useful in determining whether treatment was successful (e.g., whether a pulmonary vein 1611 was successfully electrically isolated from the left atrium of the patient's heart 1610). In some embodiments, the method 1850 can use one or more of the various parameters to inform and/or adjust energy delivered at block 1852.

At block 1854, the method 1850 can selectively display various visual indicia. In some embodiments, the method 1850 can display a model of the anatomical structure. In these and other embodiments, the method 1850 can display an icon representing the position, shape, and/or orientation of the tip section 124 and/or the shaft 122 (e.g., along or within the model of the anatomical structure). In these and still other embodiments, the method 1850 can selectively display various other information, including electrograms and/or temperature measurements captured by one or more of the sensors 826, annotations indicating areas of energy delivery (e.g., on the model of the anatomical structure, on the icon of the tip section to indicate which panel of the modular electrode 352 is currently driven, on one or more icons representing one or more previous locations of energy delivery with the modular electrode 352, etc.), and/or current parameters (e.g., amps, voltage, pulse parameters, frequency, activation time, etc.) of energy delivered to the patient 102. In these and other embodiments, the method 1850 can include displaying information pertaining to the position (e.g. relative distance, relative axial translation, relative roll, etc.) of the modular electrode 352 as compared to previous (e.g., all previous, the nearest previous, the most recent previous) energy deliveries. This can aid the physician in placing subsequent energy deliveries to ensure contiguous ablation lesions across the desired treatment area.

At block 1855, the tip section 124 can be repositioned within and/or removed from the anatomical structure of the patient 102. For example, the tip section 124 can be repositioned relative to the target pulmonary vein 1611 (e.g. more ostial or antral, or rotated with respect to the pulmonary vein 1611). As another example, the tip section 124 can be repositioned at another pulmonary vein (e.g., after successful electrical isolation of a first pulmonary vein 1611). As yet another example, the tip section 124 can be removed from the anatomical structure after completion of diagnosis and/or treatment of tissue at the treatment site. In some embodiments, the expandable portion 250 of the tip section 124 can be compressed before the tip section 124 is repositioned and/or removed. In these embodiments, the expandable portion 250 can be compressed via distal movement of the deployment member 235 relative to the distal end portion 232 of the shaft. In the case of repositioning the tip section 124 after compressing the expandable portion 250, the expandable portion 250 can be re-deployed via proximal movement of the deployment member 235 relative to the distal end portion 232 of the shaft 122.

Although the steps of the method 1850 are discussed and illustrated in a particular order, the method 1850 illustrated in FIG. 18 is not so limited. In other embodiments, the method 1850 can be performed in a different order. In these and other embodiments, any of the steps of the method 1850 can be performed before, during, and/or after any of the other steps of the method 1850. Moreover, a person of ordinary skill in the relevant art will recognize that the illustrated method can be altered and still remain within these and other embodiments of the present technology. For example, one or more steps of the method 1850 illustrated in FIG. 18 can be omitted and/or repeated in some embodiments.

### C. Additional Examples

Several aspects of the present technology are set forth in the following examples.

1. A catheter, comprising:
a shaft having a proximal end portion and a distal end portion; and
a tip section mechanically coupled to the distal end portion of the shaft, wherein the tip section includes a plurality of mesh electrode panels that together define an expandable portion.

2. The catheter of example 1 wherein the mesh electrode panels each comprise (i) a first insulated portion and a second insulated portion distributed axially along the mesh electrode panel and (ii) an active portion between the first and the second insulated portions.

3. The catheter of example 1 wherein the mesh electrode panels each comprise (i) a first insulated portion and (ii) an active portion distal the first insulated portion.

4. The catheter of any one of examples 1-3 wherein the mesh electrode panels each comprise a plurality of struts, and wherein a first subset of the plurality of struts define a plurality of cells.

5. The catheter of example 4 wherein the plurality of cells define, at least in part, an open area of the expandable portion through which fluid, blood, or a combination thereof can flow.

6. The catheter of any one of examples 1-5 wherein the tip section further includes a deployment member mechanically coupled to the expandable portion at a distalmost portion of the tip section, and wherein the expandable portion envelops at least a portion of the deployment member between the distal end portion of the shaft and the distalmost portion of the tip section.

7. The catheter of example 6 wherein the deployment member is telescoping.

8. The catheter of example 6 or example 7 wherein the expandable portion is configured to expand and compress via proximal and distal movement, respectively, of the deployment member along an axis defined by the shaft.

9. The catheter of any one of examples 6-8 wherein the deployment member defines a lumen configured to receive a guidewire.

10. The catheter of any one of examples 6-9 wherein the deployment member defines a lumen, and wherein the lumen is configured to transport fluid at least between the proximal end portion of the shaft and the distalmost portion of the tip section.

11. The catheter of any one of examples 6-10 wherein the deployment member defines a lumen and includes a plurality of holes configured to disperse fluid radially from within the expandable portion toward an inner surface of the expandable portion.

12. The catheter of any one of examples 6-11 wherein the deployment member includes at least one ring electrode positioned on the portion of the deployment member between the distal end portion of the shaft and the distalmost portion of the tip section.

13. The catheter of any one of examples 1-12 wherein:
the expandable portion is pear- or onion-shaped and includes an insulated neck portion and an active body portion distal the insulated neck portion;
the active body portion includes a modular electrode; and
the insulated neck portion is mechanically coupled to the distal end portion of the shaft.

14. The catheter of example 13 wherein:
the expandable portion further includes a nose portion distal the active body portion and the insulated neck portion; and
the nose portion is mechanically coupled to a distalmost portion of the tip section.

15. The catheter of any one of examples 1-14 wherein the mesh electrode panels each include at least one eyelet, and wherein at least one fastener holds adjacent mesh electrode panels of the expandable portion together via the corresponding eyelets.

16. The catheter of example 15 wherein the at least one fastener includes at least one sensor, and/or wherein the at least one sensor includes at least one electrode and/or a temperature measurement device.

17. The catheter of example 16 wherein the at least one eyelet of each of the mesh electrode panels is directly connected to at least one strut, and wherein the at least one strut includes a bend such that the at least one sensor is recessed relative to an exterior of the expandable portion when the adjacent mesh electrode panels are held together via the at least one fastener.

18. The catheter of example 16 or example 17 wherein an electrical lead extends from the at least one sensor, within an interior of the expandable portion, and into the shaft, and wherein a sensor is formed by and/or is positioned on the electrical lead within the interior of the expandable portion.

19. The catheter of any one of examples 1-18 further comprising a displacement measuring device configured to measure a displacement of a deployment member mechanically coupled to the expandable portion to determine a shape of the expandable portion.

20. The catheter of any one of examples 1-19 further comprising a shaft electrode mounted to the distal end portion of the shaft.

21. The catheter of any one of examples 1-20 wherein the mesh electrode panels of the expandable portion are electrically isolated from one another such that electrical energy can be delivered from any one of the mesh electrode panels independently from the other of the mesh electrode panels.

22. The catheter of any one of examples 1-21 wherein:
each of the mesh electrode panels includes a keyed portion at a proximal end and/or at a distal end of the mesh electrode panel;
the keyed portions are configured to interface with a first coupler at the distal end portion of the shaft and/or a second coupler at a distalmost portion of the tip section; and
the keyed portions and the first and/or second couplers are configured to mechanically couple the mesh electrode panels to the distal end portion of the shaft and/or to the distalmost portion of the tip section.

23. The catheter of any one of examples 1-22 wherein at least one of the mesh electrode panels includes:
a first strut mechanically coupled to a deployment member at a distalmost portion of the tip section;
a second strut coupled to the first strut at a distalmost portion of the second strut; and
a third strut coupled to the first strut at a distalmost portion of the third strut.

24. The catheter of any one of examples 1-23 wherein:
each of the mesh electrode panels includes at least one strut mechanically coupled to a deployment member at a distalmost portion of the tip section; and
the at least one strut of each of the mesh electrode panels emerges distally from the distalmost portion of the tip section.

25. The catheter of any one of examples 1-24 wherein at least one of the mesh electrode panels includes:
a first strut mechanically coupled to the distal end portion of the shaft;
a second strut coupled to the first strut at a proximal-most portion of the second strut; and
a third strut coupled to the first strut at a proximal-most portion of the third strut.

26. The catheter of any one of examples 1-25 wherein at least one mesh electrode panel includes a proximal portion, a distal portion, and a middle portion between the proximal portion and the distal portion, and wherein the middle portion is wider than the proximal and the distal portion.

27. The catheter of any one of examples 1-26 wherein:
the mesh electrode panels each comprise a plurality of struts;
a first subset of the plurality of struts defines a plurality of cells of the expandable portion;
the expandable portion includes a distal section, a proximal section, and an equator between the distal section and the proximal section; and
the expandable portion includes a greater number of cells of the plurality of cells about the equator than about the distal section and/or about the proximal section.

28. The catheter of any one of examples 1-27 wherein:
the mesh electrode panels each comprise a plurality of struts;
a first subset of the plurality of struts defines a plurality of cells of the expandable portion; and
at least one cell of the plurality of cells is formed by (i) a first strut of the first subset belonging to a first one of the mesh electrode panels and (ii) a second strut of the first subset belonging to a second one of the mesh electrode panels different from the first one.

29. The catheter of any one of examples 1-28 wherein:
the mesh electrode panels each comprise a plurality of struts;
a first subset of the plurality of struts defines a plurality of cells of the expandable portion; and
a distalmost cell of the plurality of cells is formed by (i) a first strut of the first subset belonging to a first one of the mesh electrode panels and (ii) a second strut of the first subset belonging to a second one of the mesh electrode panels different from the first one.

30. The catheter of any one of examples 1-29 wherein:
the mesh electrode panels each comprise a plurality of struts;
a first subset of the plurality of struts defines a plurality of cells of the expandable portion; and
a proximal-most cell of the plurality of cells is formed by (i) a first strut of the first subset belonging to a first one of the mesh electrode panels and (ii) a second strut of the first subset belonging to a second one of the mesh electrode panels different from the first one.

31. The catheter of any one of examples 1-30 wherein:
the electrode panels each comprise a plurality of struts;
the plurality of struts includes (i) a first subset of struts having one or more first lengths and/or one or more first widths and (ii) a second subset of struts having one or more second lengths smaller than the one or more first lengths and/or one or more seconds widths smaller than the one or more first widths; and
the first subset of struts includes a distalmost strut of the plurality of struts and/or a proximal-most strut of the plurality of struts.

32. The catheter of any one of examples 1-31 wherein:
the mesh electrode panels each comprise a plurality of struts;
the plurality of struts defines a plurality of cells of the expandable portion; and
at least one cell of the plurality of cells is formed by at least four struts.

33. The catheter of any one of examples 1-32 wherein:
at least one mesh electrode panel comprises a plurality of struts; and
a proximal-most strut of the plurality of struts includes a first portion having a first width and a second portion having a second width smaller than the first width.

34. The catheter of any one of examples 1-33 wherein the tip section further includes at least one location coil sensor configured to measure positional and/or pose information of the tip section.

35. The catheter of any one of examples 1-34 wherein, in the absence of external force, the expandable portion assumes a deployed state having a diameter greater than a largest diameter of the shaft.

36. A method for treating target tissue at a treatment site within a patient using a tip section of a catheter, the method comprising:
determining an effective surface area of the tip section of the catheter, wherein the tip section includes a plurality of mesh electrode panels, and wherein the mesh electrode panels of the plurality are electrically insulated from one another and together define an expandable portion of the tip section; and
delivering energy to the target tissue at the treatment site,
wherein the energy is delivered via at least one mesh electrode panel of the expandable portion based, at least in part, on the determined effective surface area.

37. The method of example 36 wherein determining the effective surface area includes determining a position and/or orientation of the tip section.

38. The method of example 37 wherein determining the position and/or the orientation of the tip section includes:
fluoroscopically visualizing the tip section; and/or
receiving at least one signal from a location coil sensor of the tip section, wherein the at least one signal is indicative of the position of the location coil sensor in three-dimensional space and/or is indicative of pitch, yaw, and/or roll of the location coil sensor.

39. The method of any one of examples 36-37 wherein delivering the energy to the at least one mesh electrode panel of the expandable portion includes delivering the energy to the at least one mesh electrode panel based at least in part on a position of the at least one mesh electrode panel within an anatomical structure of the patient.

40. The method of any one of examples 36-39 wherein determining the effective surface area includes determining an extent of deployment and/or deformation of the expandable portion.

41. The method of example 40 wherein determining the extent of deployment and/or deformation of the expandable portion includes:
fluoroscopically visualizing the tip section; and/or
receiving one or more signals from two or more electrodes mounted on the expandable portion, wherein the one or more signals are indicative of impedance between the two or more electrodes.

42. The method of any one of examples 36-41 wherein determining the effective surface area include determining an extent of contact of the at least one mesh electrode panel with tissue.

43. The method of any one of examples 36-42 wherein delivering the energy to the at least one mesh electrode panel of the expandable portion includes delivering a specified amount of energy to the at least one mesh electrode panel to achieve a target current density of energy delivered through the at least one mesh electrode panel to tissue in contact with the at least one mesh electrode panel.

44. The method of any one of examples 36-43 wherein delivering the energy to the at least one mesh electrode panel includes delivering the energy to all of the mesh electrode panels of the expandable portion.

45. The method of example 44 wherein delivering the energy to all of the mesh electrode panels of the expandable portion includes separately and sequentially delivering the energy to individual mesh electrode panels of the expandable portion.

46. The method of example 44 wherein delivering the energy to all of the mesh electrode panels of the expandable portion includes separately and sequentially delivering the energy to subgroupings of the mesh electrode panels of the expandable portion.

47. The method of any one of examples 36-43 wherein the at least one mesh electrode panel includes a subset of the mesh electrode panels of the expandable portion, wherein the subset includes less than all of the mesh electrode panels of the expandable portion, and further wherein delivering the energy to the at least one mesh electrode panel includes delivering the energy to only the mesh electrode panels of the subset.

48. The method of any one of examples 36-47 wherein delivering the energy to the at least one mesh electrode panel includes delivering radiofrequency (RF) energy and/or pulsed field energy to the at least one mesh electrode panel.

49. The method of any one of examples 36-48, further comprising:
receiving one or more signals from one or more temperature measurement devices mounted on the expandable portion, wherein the one or more signals are indicative of temperatures of tissue in contact with the at least one mesh electrode panel;
adjusting the energy delivered to the at least one mesh electrode panel based at least in part on the received one or more temperature signals; and/or
delivering irrigation fluid to the tissue in contact with the least one mesh electrode panel.

50. An electrode panel for use in forming an expandable portion of a tip section of a catheter, the electrode panel comprising:
a first section at a proximal end portion of the electrode panel;
a second section at a distal end portion of the electrode panel; and
an active section between the first and second sections.

51. The electrode panel of example 50 wherein the first section is insulated.

52. The electrode panel of example 50 or example 51 wherein at least a portion of the second section is insulated.

53. The electrode panel of any one of examples 50-52 wherein:
the active section includes a plurality of struts; and
the plurality of struts define a plurality of cells.

54. The electrode panel of example 53 wherein the plurality of cells define, at least in part, an open area of the electrode panel through which fluid, blood, or a combination thereof can flow.

55. The electrode panel of example 53 or example 54 wherein at least one cell of the plurality of cells is defined by at least four struts of the plurality of struts.

56. The electrode panel of any one of examples 50-55 wherein:
the active section includes a plurality of struts;
the first section includes at least one strut;
the at least one strut of the first section has a first length and/or a first width; and
struts of the plurality of struts have second lengths smaller than the first length and/or second widths smaller than the first width.

57. The electrode panel of any one of examples 50-56 wherein:
the active section includes a plurality of struts;
the second section includes at least one strut;
the at least one strut of the second section has a first length and/or a first width; and
struts of the plurality of struts have second lengths smaller than the first length and/or second widths smaller than the first width.

58. The electrode panel of any one of examples 50-57 wherein:
the first section includes at least one strut; and
the at least one strut includes a first portion having a first width and a second portion having a second width smaller than the first width.

59. The electrode panel of any one of examples 50-58 wherein the active section is wider than the first section.

60. The electrode panel of any one of examples 50-59 wherein the active section is wider than the second section.

61. The electrode panel of any one of examples 50-60 wherein the first section includes a single strut.

62. The electrode panel of any one of examples 50-61 wherein:
the first section includes a single strut;
the active section includes a first strut directly coupled to the single strut at a proximal-most portion of the first strut; and
the active section further includes a second strut directly coupled to the single strut at a proximal-most portion of the second strut.

63. The electrode panel of any one of examples 50-62 wherein:
the first section includes a single strut;
the active section includes a first strut directly coupled to the single strut at a distal end of the single strut; and
the active section includes a second strut directly coupled to the single strut at the distal end of the single strut.

64. The electrode panel of any one of examples 50-63 wherein the second section includes a single strut.

65. The electrode panel of any one of examples 50-64 wherein:
the second section includes a single strut;
the active section includes a first strut directly coupled to the single strut at a distalmost portion of the first strut; and
the active section further includes a second strut directly coupled to the single strut at a distalmost portion of the second strut.

66. The electrode panel of any one of examples 50-65 wherein:
the second section includes a single strut;
the active section includes a first strut directly coupled to the single strut at a proximal end of the single strut; and
the active section includes a second strut directly coupled to the single strut at the proximal end of the single strut.

67. The electrode panel of any one of examples 50-66 wherein the second section includes at least one strut, and wherein a strut of the at least one strut is configured to be mechanically coupled to a distalmost portion of a tip section of a catheter.

68. The electrode panel of example 67 wherein the strut of the at least one strut is configured to emerge distally from the distalmost portion of the tip section when mechanically coupled to the distalmost portion of the tip section.

69. The electrode panel of any one of examples 50-68 wherein:
the electrode panel includes a keyed portion at a distal end of the electrode panel;
the keyed portion is configured to interface with a coupler at a distalmost portion of a tip section of a catheter; and
the keyed portion and the coupler are configured to mechanically couple the electrode panel to the distalmost portion of the tip section.

70. The electrode panel of any one of examples 50-69 wherein the first section includes at least one strut, and wherein a strut of the at least one strut is configured to be mechanically coupled to a distal end portion of a catheter shaft.

71. The electrode panel of any one of examples 50-70 wherein:
the electrode panel includes a keyed portion at a proximal end of the electrode panel;
the keyed portion is configured to interface with a coupler at a distal end portion of a catheter shaft; and
the keyed portion and the coupler are configured to mechanically couple the electrode panel to the distal end portion of the catheter shaft.

72. The electrode panel of any one of examples 50-71 further comprising at least one eyelet configured to receive a fastener such that the electrode panel can be mechanically coupled to another electrode panel.

73. The electrode panel of example 72 wherein:
the active section includes a plurality of struts; and
the at least one eyelet is directly connected to at least one strut of the plurality of struts.

74. The electrode panel of example 73 wherein the at least one strut includes a bend such that the eyelet is not flush with other struts of the plurality of struts.

75. The electrode panel of any one of examples 50-74 wherein the second section is not insulated.

### D. Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. Furthermore, the various embodiments described herein can also be combined to provide further embodiments.

The systems and methods described herein can be provided in the form of tangible and non-transitory machine-readable medium or media (such as a hard disk drive, hardware memory, etc.) having instructions recorded thereon for execution by a processor or computer. The set of instructions can include various commands that instruct the computer or processor to perform specific operations such as the methods and processes of the various embodiments described here. The set of instructions can be in the form of a software program or application. The computer storage media can include volatile and non-volatile media, and removable and non-removable media, for storage of information such as computer-readable instructions, data structures, program modules or other data. The computer storage media can include, but are not limited to, RAM, ROM, EPROM, EEPROM, flash memory or other solid-state memory technology, CD-ROM, DVD, or other optical storage, magnetic disk storage, or any other hardware medium which can be used to store desired information and that can be accessed by components of the system. Components of the system can communicate with each other via wired or wireless communication. The components can be separate from each other, or various combinations of components can be integrated together into a monitor or processor or contained within a workstation with standard computer hardware (for example, processors, circuitry, logic circuits, memory, and the like). The system can include processing devices such as microprocessors, microcontrollers, integrated circuits, control units, storage media, and other hardware.

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and both A and B. Additionally, the terms “comprising,” “including,” “having” and “with” are used throughout to mean including at least the recited feature(s) such that any greater number of the same feature and/or additional types of other features are not precluded. Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

From the foregoing, it will also be appreciated that various modifications can be made without deviating from the technology. For example, various components of the technology can be further divided into subcomponents, or various components and functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

I/We claim:

1. A catheter, comprising:
a shaft having a proximal end portion and a distal end portion; and
a tip section mechanically coupled to the distal end portion of the shaft, wherein the tip section includes a plurality of mesh electrode panels that together define an expandable portion.

2. The catheter of claim 1 wherein the mesh electrode panels each comprise (i) a first insulated portion and a second insulated portion distributed axially along the mesh electrode panel and (ii) an active portion between the first and the second insulated portions.

3. The catheter of claim 1 wherein the mesh electrode panels each comprise (i) a first insulated portion and (ii) an active portion distal the first insulated portion.

4. The catheter of claim 1 wherein the mesh electrode panels each comprise a plurality of struts, and wherein a first subset of the plurality of struts define a plurality of cells.

5. The catheter of claim 4 wherein the plurality of cells define, at least in part, an open area of the expandable portion through which fluid, blood, or a combination thereof can flow.

6. The catheter of claim 1 wherein the tip section further includes a deployment member mechanically coupled to the expandable portion at a distalmost portion of the tip section, and wherein the expandable portion envelops at least a portion of the deployment member between the distal end portion of the shaft and the distalmost portion of the tip section.

7. The catheter of claim 6 wherein the deployment member is telescoping.

8. The catheter of claim 6 wherein the expandable portion is configured to expand and compress via proximal and distal movement, respectively, of the deployment member along an axis defined by the shaft.

9. The catheter of claim 6 wherein the deployment member defines a lumen configured to receive a guidewire.

10. The catheter of claim 6 wherein the deployment member defines a lumen, and wherein the lumen is configured to transport fluid at least between the proximal end portion of the shaft and the distalmost portion of the tip section.

11. The catheter of claim 6 wherein the deployment member defines a lumen and includes a plurality of holes configured to disperse fluid radially from within the expandable portion toward an inner surface of the expandable portion.

12. The catheter of claim 6 wherein the deployment member includes at least one ring electrode positioned on the portion of the deployment member between the distal end portion of the shaft and the distalmost portion of the tip section.

13. The catheter of claim 1 wherein:
the expandable portion is pear- or onion-shaped and includes an insulated neck portion and an active body portion distal the insulated neck portion;
the active body portion includes a modular electrode; and
the insulated neck portion is mechanically coupled to the distal end portion of the shaft.

14. The catheter of claim 13 wherein:
the expandable portion further includes a nose portion distal the active body portion and the insulated neck portion; and
the nose portion is mechanically coupled to a distalmost portion of the tip section.

15. The catheter of claim 1 wherein the mesh electrode panels each include at least one eyelet, and wherein at least one fastener holds adjacent mesh electrode panels of the expandable portion together via the corresponding eyelets.

16. The catheter of claim 15 wherein the at least one fastener includes at least one sensor, and/or wherein the at least one sensor includes at least one electrode and/or a temperature measurement device.

17. The catheter of claim 16 wherein the at least one eyelet of each of the mesh electrode panels is directly connected to at least one strut, and wherein the at least one strut includes a bend such that the at least one sensor is recessed relative to an exterior of the expandable portion when the adjacent mesh electrode panels are held together via the at least one fastener.

18. The catheter of claim 16 wherein an electrical lead extends from the at least one sensor, within an interior of the expandable portion, and into the shaft, and wherein a sensor is formed by and/or is positioned on the electrical lead within the interior of the expandable portion.

19. The catheter of claim 1, further comprising a displacement measuring device configured to measure a displacement of a deployment member mechanically coupled to the expandable portion to determine a shape of the expandable portion.

20. The catheter of claim 1, further comprising a shaft electrode mounted to the distal end portion of the shaft.

21. The catheter of claim 1 wherein the mesh electrode panels of the expandable portion are electrically isolated from one another such that electrical energy can be delivered from any one of the mesh electrode panels independently from the other of the mesh electrode panels.

22. The catheter of claim 1 wherein:
each of the mesh electrode panels includes a keyed portion at a proximal end and/or at a distal end of the mesh electrode panel;
the keyed portions are configured to interface with a first coupler at the distal end portion of the shaft and/or a second coupler at a distalmost portion of the tip section; and
the keyed portions and the first and/or second couplers are configured to mechanically couple the mesh electrode panels to the distal end portion of the shaft and/or to the distalmost portion of the tip section.

23. The catheter of claim 1 wherein at least one mesh electrode panel includes:
a first strut mechanically coupled to a deployment member at a distalmost portion of the tip section;
a second strut coupled to the first strut at a distalmost portion of the second strut; and
a third strut coupled to the first strut at a distalmost portion of the third strut.

24. The catheter of claim 1 wherein:
each of the mesh electrode panels includes at least one strut mechanically coupled to a deployment member at a distalmost portion of the tip section; and
the at least one strut of each of the mesh electrode panels emerges distally from the distalmost portion of the tip section.

25. The catheter of claim 1 wherein at least one of the mesh electrode panels includes:
a first strut mechanically coupled to the distal end portion of the shaft;
a second strut coupled to the first strut at a proximal-most portion of the second strut; and
a third strut coupled to the first strut at a proximal-most portion of the third strut.

26. The catheter of claim 1 wherein at least one mesh electrode panel includes a proximal portion, a distal portion, and a middle portion between the proximal portion and the distal portion, and wherein the middle portion is wider than the proximal and the distal portion.

27. The catheter of claim 1 wherein:
the mesh electrode panels each comprise a plurality of struts;
a first subset of the plurality of struts defines a plurality of cells of the expandable portion;
the expandable portion includes a distal section, a proximal section, and an equator between the distal section and the proximal section; and
the expandable portion includes a greater number of cells of the plurality of cells about the equator than about the distal section and/or about the proximal section.

28. The catheter of claim 1 wherein:
the mesh electrode panels each comprise a plurality of struts;
a first subset of the plurality of struts defines a plurality of cells of the expandable portion; and
at least one cell of the plurality of cells is formed by (i) a first strut of the first subset belonging to a first one of the mesh electrode panels and (ii) a second strut of the first subset belonging to a second one of the mesh electrode panels different from the first one.

29. The catheter of claim 1 wherein:
the mesh electrode panels each comprise a plurality of struts;
a first subset of the plurality of struts defines a plurality of cells of the expandable portion; and
a distalmost cell of the plurality of cells is formed by (i) a first strut of the first subset belonging to a first one of the mesh electrode panels and (ii) a second strut of the first subset belonging to a second one of the mesh electrode panels different from the first one.

30. The catheter of claim 1 wherein:
the mesh electrode panels each comprise a plurality of struts;
a first subset of the plurality of struts defines a plurality of cells of the expandable portion; and
a proximal-most cell of the plurality of cells is formed by (i) a first strut of the first subset belonging to a first one of the mesh electrode panels and (ii) a second strut of the first subset belonging to a second one of the mesh electrode panels different from the first one.

31. The catheter of claim 1 wherein:
the electrode panels each comprise a plurality of struts;
the plurality of struts includes (i) a first subset of struts having one or more first lengths and/or one or more first widths and (ii) a second subset of struts having one or more second lengths smaller than the one or more first lengths and/or one or more seconds widths smaller than the one or more first widths; and
the first subset of struts includes a distalmost strut of the plurality of struts and/or a proximal-most strut of the plurality of struts.

32. The catheter of claim 1 wherein:
the mesh electrode panels each comprise a plurality of struts;
the plurality of struts defines a plurality of cells of the expandable portion; and
at least one cell of the plurality of cells is formed by at least four struts.

33. The catheter of claim 1 wherein:
at least one mesh electrode panel comprises a plurality of struts; and
a proximal-most strut of the plurality of struts includes a first portion having a first width and a second portion having a second width smaller than the first width.

34. The catheter of claim 1 wherein the tip section further includes at least one location coil sensor configured to measure positional and/or pose information of the tip section.

35. The catheter of claim 1 wherein, in the absence of external force, the expandable portion assumes a deployed state having a diameter greater than a largest diameter of the shaft.

36. A method for treating target tissue at a treatment site within a patient using a tip section of a catheter, the method comprising:
determining an effective surface area of the tip section of the catheter, wherein the tip section includes a plurality of mesh electrode panels, and wherein the mesh electrode panels of the plurality are electrically insulated from one another and together define an expandable portion of the tip section; and
delivering energy to the target tissue at the treatment site,
wherein the energy is delivered via at least one mesh electrode panel of the expandable portion based, at least in part, on the determined effective surface area.

37. The method of claim 36 wherein determining the effective surface area includes determining a position and/or orientation of the tip section.

38. The method of claim 37 wherein determining the position and/or the orientation of the tip section includes:
fluoroscopically visualizing the tip section; and/or
receiving at least one signal from a location coil sensor of the tip section, wherein the at least one signal is indicative of the position of the location coil sensor in three-dimensional space and/or is indicative of pitch, yaw, and/or roll of the location coil sensor.

39. The method of claim 36 wherein delivering the energy to the at least one mesh electrode panel of the expandable portion includes delivering the energy to the at least one mesh electrode panel based at least in part on a position of the at least one mesh electrode panel within an anatomical structure of the patient.

40. The method of claim 36 wherein determining the effective surface area includes determining an extent of deployment and/or deformation of the expandable portion.

41. The method of claim 40 wherein determining the extent of deployment and/or deformation of the expandable portion includes:
fluoroscopically visualizing the tip section; and/or
receiving one or more signals from two or more electrodes mounted on the expandable portion, wherein the one or more signals are indicative of impedance between the two or more electrodes.

42. The method of claim 36 wherein determining the effective surface area include determining an extent of contact of the at least one mesh electrode panel with tissue.

43. The method of claim 36 wherein delivering the energy to the at least one mesh electrode panel of the expandable portion includes delivering a specified amount of energy to the at least one mesh electrode panel to achieve a target current density of energy delivered through the at least one mesh electrode panel to tissue in contact with the at least one mesh electrode panel.

44. The method of claim 36 wherein delivering the energy to the at least one mesh electrode panel includes delivering the energy to all of the mesh electrode panels of the expandable portion.

45. The method of claim 44 wherein delivering the energy to all of the mesh electrode panels of the expandable portion includes separately and sequentially delivering the energy to individual mesh electrode panels of the expandable portion.

46. The method of claim 44 wherein delivering the energy to all of the mesh electrode panels of the expandable portion includes separately and sequentially delivering the energy to subgroupings of the mesh electrode panels of the expandable portion.

47. The method of claim 36 wherein the at least one mesh electrode panel includes a subset of the mesh electrode panels of the expandable portion, wherein the subset includes less than all of the mesh electrode panels of the expandable portion, and further wherein delivering the energy to the at least one mesh electrode panel includes delivering the energy to only the mesh electrode panels of the subset.

48. The method of claim 36 wherein delivering the energy to the at least one mesh electrode panel includes delivering radiofrequency (RF) energy and/or pulsed field energy to the at least one mesh electrode panel.

49. The method of claim 36, further comprising:
receiving one or more signals from one or more temperature measurement devices mounted on the expandable portion, wherein the one or more signals are indicative of temperatures of tissue in contact with the at least one mesh electrode panel;
adjusting the energy delivered to the at least one mesh electrode panel based at least in part on the received one or more temperature signals; and/or
delivering irrigation fluid to the tissue in contact with the least one mesh electrode panel.

50. An electrode panel for use in forming an expandable portion of a tip section of a catheter, the electrode panel comprising:
a first section at a proximal end portion of the electrode panel;
a second section at a distal end portion of the electrode panel; and
an active section between the first and second sections.

51. The electrode panel of claim 50 wherein the first section is insulated.

52. The electrode panel of claim 50 wherein at least a portion of the second section is insulated.

53. The electrode panel of claim 50 wherein:
the active section includes a plurality of struts; and
the plurality of struts define a plurality of cells.

54. The electrode panel of claim 53 wherein the plurality of cells define, at least in part, an open area of the electrode panel through which fluid, blood, or a combination thereof can flow.

55. The electrode panel of claim 53 wherein at least one cell of the plurality of cells is defined by at least four struts of the plurality of struts.

56. The electrode panel of claim 50 wherein:
the active section includes a plurality of struts;
the first section includes at least one strut;
the at least one strut of the first section has a first length and/or a first width; and
struts of the plurality of struts have second lengths smaller than the first length and/or second widths smaller than the first width.

57. The electrode panel of claim 50 wherein:
the active section includes a plurality of struts;
the second section includes at least one strut;
the at least one strut of the second section has a first length and/or a first width; and
struts of the plurality of struts have second lengths smaller than the first length and/or second widths smaller than the first width.

58. The electrode panel of claim 50 wherein:
the first section includes at least one strut; and
the at least one strut includes a first portion having a first width and a second portion having a second width smaller than the first width.

59. The electrode panel of claim 50 wherein the active section is wider than the first section.

60. The electrode panel of claim 50 wherein the active section is wider than the second section.

61. The electrode panel of claim 50 wherein the first section includes a single strut.

62. The electrode panel of claim 50 wherein:
the first section includes a single strut;
the active section includes a first strut directly coupled to the single strut at a proximal-most portion of the first strut; and
the active section further includes a second strut directly coupled to the single strut at a proximal-most portion of the second strut.

63. The electrode panel of claim 50 wherein:
the first section includes a single strut;
the active section includes a first strut directly coupled to the single strut at a distal end of the single strut; and
the active section includes a second strut directly coupled to the single strut at the distal end of the single strut.

64. The electrode panel of claim 50 wherein the second section includes a single strut.

65. The electrode panel of claim 50 wherein:
the second section includes a single strut;
the active section includes a first strut directly coupled to the single strut at a distalmost portion of the first strut; and
the active section further includes a second strut directly coupled to the single strut at a distalmost portion of the second strut.

66. The electrode panel of claim 50 wherein:
the second section includes a single strut;
the active section includes a first strut directly coupled to the single strut at a proximal end of the single strut; and
the active section includes a second strut directly coupled to the single strut at the proximal end of the single strut.

67. The electrode panel of claim 50 wherein the second section includes at least one strut, and wherein a strut of the at least one strut is configured to be mechanically coupled to a distalmost portion of a tip section of a catheter.

68. The electrode panel of claim 67 wherein the strut of the at least one strut is configured to emerge distally from the distalmost portion of the tip section when mechanically coupled to the distalmost portion of the tip section.

69. The electrode panel of claim 50 wherein:
the electrode panel includes a keyed portion at a distal end of the electrode panel;
the keyed portion is configured to interface with a coupler at a distalmost portion of a tip section of a catheter; and
the keyed portion and the coupler are configured to mechanically couple the electrode panel to the distalmost portion of the tip section.

70. The electrode panel of claim 50 wherein the first section includes at least one strut, and wherein a strut of the at least one strut is configured to be mechanically coupled to a distal end portion of a catheter shaft.

71. The electrode panel of claim 50 wherein:
the electrode panel includes a keyed portion at a proximal end of the electrode panel;
the keyed portion is configured to interface with a coupler at a distal end portion of a catheter shaft; and
the keyed portion and the coupler are configured to mechanically couple the electrode panel to the distal end portion of the catheter shaft.

72. The electrode panel of claim 50 further comprising at least one eyelet configured to receive a fastener such that the electrode panel can be mechanically coupled to another electrode panel.

73. The electrode panel of claim 72 wherein:
the active section includes a plurality of struts; and
the at least one eyelet is directly connected to at least one strut of the plurality of struts.

74. The electrode panel of claim 73 wherein the at least one strut includes a bend such that the eyelet is not flush with other struts of the plurality of struts.

75. The electrode panel of claim 50 wherein the second section is not insulated.
